(19) United States
(12) Patent Application Publication
(10) Pub. No.: US 2012/0172944 A1
(43) Pub. Date: Jul. 5, 2012

(54) METHODS AND APPARATUS FOR OPTIMIZING CARDIAC OUTPUT, PREVENTING BACKWARD HEART FAILURE, AND MINIMIZING DIASTOLIC MYOCARDIAL WALL STRESS BY CONTROLLING LEFT VENTRICULAR FILLING

(75) Inventors: Guy Dori, Haifa (IL); Oscar Lichtenstein, Haifa (IL)

(73) Assignee: D.H.S.MEDICAL LTD., Nazareth (IL)

(21) Appl. No.: 13/394,574

(22) PCT Filed: Sep. 7, 2010
(86) PCT No.: PCT/IB10/54023

§ 371 Date: Mar. 7, 2012

Related U.S. Application Data
(60) Provisional application No. 61/240,846, filed on Sep. 9, 2009.

(51) Int. Cl. A61N 1/365; A61N 1/39

(52) U.S. Cl. 607/6; 607/24; 607/18

(57) ABSTRACT

Apparatus for diastole trimming including a controller for producing a diastole ending signal, and one or more leads connected to the controller, for carrying the signal to lead connections to a heart, characterized by the controller detecting when a left ventricle (LV) of the heart is mostly full, and producing the diastole ending signal such that the diastole duration is trimmed. Apparatus for diastole trimming including a controller for producing a diastole ending signal, and a connection to a pacemaker, characterized by the controller having decision rules for indicating to the pacemaker when to fire and end the diastole. A method of programming a pacemaker characterized by increasing cardiac output by trimming duration of diastole. A method for increasing cardiac output including producing a signal to trim diastole duration, thereby increasing heart rate (HR) and increasing a product of stroke volume (SV) times HR. Related apparatus and methods are also described.

### RELATED APPLICATION/S

This application is a PCT application claiming priority from U.S. Provisional Patent Application No. 61/240,846, of Dori, filed on 9 Sep. 2009.

The contents of the above document are incorporated by reference as if fully set forth herein.

### FIELD AND BACKGROUND OF THE INVENTION

The present invention, in some embodiments thereof, relates to a method and apparatus for optimizing cardiac performance by controlling time of left ventricular filling, and, more particularly, but not exclusively, to the treatment of heart failure, to apparatuses and methods for treatment of diastolic heart failure, to improving right ventricle output, to preventing backward heart failure and minimizing diastolic myocardial wall stress, in patients with diastolic heart failure. The invention may also be used in other clinical contexts as described below.

Heart failure (HF) is a complex clinical syndrome. In about half of the cases of HF the left ventricle (LV) fails to eject blood to the circulation (termed: systolic HF), and in the other half, the LV fails to relax and distend and fill with blood (termed: diastolic HF or DHF).

Worldwide there are 23 million people with HF. Approximately half of all HF is due to DHF. In the US, at any one time, there are 3-6 million patients with DHF, and 0.33-0.66 million new cases of DHF every year, according to www.americanheart.org; and Braunwald's Ed. 8, Ch. 22. In the US there were 0.5 million hospitalizations due to DHF in 2009, at a total cost of approximately 20 billion US dollars, according to “Heart disease and stroke statistics 2010 update”, p. 20. This huge cohort of patients grows with the increasing percentage of the elderly, greater than 65 years of age, in the population. In 2000, there were 35 million elderly in the US, where this number is expected to double in 2030 to 70 million.

The number of patients with heart failure (HF) increases throughout the world and poses a major clinical and economic problem. It is estimated that 6-10% of the population aged greater than 65 years of age suffer from HF. 50% of heart failure is diastolic (diastolic heart failure, DHF), in which ejection fraction is preserved at greater than 50%, and yet patients suffer from the clinical syndrome of HF (dyspnea, fatigue, accumulation of fluids in the body) and at times deteriorate to life threatening conditions such as pulmonary edema, according to Vasan and Nishimura (see reference list below).

At present, the treatment of DHF is not specific, and its main goal is to reduce heart rate (HR) with drugs, such as beta-blockers, thus extending the time of diastole, allowing more time for filling of the heart with blood. Another group of drugs which is often used for treating DHF patients is diuretics. Diuretics increase urination, thus reducing the volume of blood in the blood vessels. Specific drugs which change the mechanical properties of the cardiac muscle in order to improve filling of the heart are presently not available.

Current treatment of DHF is unsatisfactory. It is mainly symptomatic and aims at controlling comorbidities such as diabetes, hypertension and hyperlipidemia. If comorbidities are present, patient is treated accordingly. If patient suffers from excess of blood volume, such as pulmonary or leg edema, diuretics are administered in order to decrease the intravascular volume.

One line of treatment of DHF is directed at the impairment of the LV to dilate and fill with blood. The line of treatment is led by heart rate (HR) slowing drugs, such as beta receptor blockers (BB), or calcium channel blockers (CCB). A physiologic rationale for this line of treatment is to allow more time for LV filling, that is, prolonging the diastole by slowing HR, thereby increasing the volume of blood filling the heart, and consequently increasing cardiac output (CO). In other words, the standard of care, using HR slowing drugs, attempts to increase CO, which is the product of HR and stroke volume by augmenting the filling of the LV.

A recent review by Das et al. of the treatment of DHF noted the following:

A. Survival improved for HF patients with reduced ejection fraction (EF), while “there was no such trend toward improvement among patients with HF and preserved EF”.

B. Two large randomized clinical studies did not achieve statistical significance in benefit of renin-angiotensin system blockade on their primary combined endpoints of morbidity and mortality. No clear benefit of beta-blocker has been demonstrated specifically in patients with DHF.

C. Current therapeutic recommendations for HF with preserved EF are aimed mostly at symptomatic management and treatment of concomitant comorbidities.

D. “There exists an urgent need to develop effective treatment strategies specifically for patients with HF and preserved EF.”

In support of the above points see also Acikel.

While treatment of HF with reduced EF has improved, the treatment of HF with preserved EF, that is DHF, awaits a much needed breakthrough.

Background art includes:
Libby P, Bonow R O, Mann D I, Zipes D P, in Braunwald's Heart Disease a textbook of cardiovascular medicine. Ed. 8th. 2008, Chapters 21 and 22. Saunders Elsevier, Phila, Pa.
Vasan R S, Benjamin E J, Levy D. Prevalence, clinical features and prognosis of diastolic heart failure: an epidemiologic perspective. JACC 1995; 26: 1565:74.
Nishimura R A, Tajik J A. Evaluation of diastolic filling of left ventricle in health and disease: Doppler echocardiography is the clinician's Rosetta stone? JACC 1997; 30:8-18.
Das A, Abraham S, Deswal A. Advances in the treatment of heart failure with preserved ejection fraction. Current opinion in cardiology 2008; 23: 233-240.
Owan et al. Trends in Prevalence and Outcome of HF with preserved EF. NEJM 2006; 355: 251-9.
Ohno M, Cheng C P, Little W C. Congestive heart failure: mechanism of altered patterns of left ventricular filling during the development of congestive heart failure. Circulation 1994; 89: 2241-2250.
Acikel S, Akdemir R, Kilic H, Yesilay A B, Dogan M, Cagirci G. Diastolic heart failure in elderly: The prognostic factors and interventions regarding heart failure with presered ejection fraction. Int J Cardiol 2008; Oct. 7 ahead of print.
Kenichi Nakajima, Junichi Taki, Masaya Kawano, Takahiro Higuchi, Shinichi Sato, Chihiro Nishijima, Kazuhiko Takehara and Norihisa Tonami. Diastolic Dysfunction in Patients with Systemic Sclerosis Detected by Gated Myocardial Perfusion SPECT: An Early Sign of Cardiac Involvement. Journal of Nuclear Medicine 2001; 42: 183-188.
Pina I l, Apstein C S, Balady G J et al Exercise and heart failure: a statement from the American Heart Association committee on exercise, rehabilitation and prevention. Circulation 2003; 107: 1210-1225.
U.S. Pat. No. 7,272,443 to Min et al;
U.S. Pat. No. 6,738,667 to Deno et al;
U.S. Pat. No. 6,650,940 to Zhu et al;
U.S. Pat. No. 6,438,408 to Mulligan et al;
U.S. Pat. No. 6,285,906 to Ben-Haim et al;
U.S. Pat. No. 6,236,887 to Ben-Haim et al;
U.S. Pat. No. 6,233,484 to Ben-Haim et al;
U.S. Pat. No. 6,078,835 to Hedberg et al;
U.S. Pat. No. 5,800,464 to Kieval;
US Patent Application number 2007/0203522 of Hettrick et al;
US Patent Application number 2007/0239219 of Salo et al;
Heart disease and stroke statistics 2010 update. American heart Association.

### SUMMARY OF THE INVENTION

The present invention, in some embodiments thereof, electronically controls timing of the heart cycle, based on noticing that, in some patients, the left ventricle (LV) fails to fully relax, distend, and fill with blood during the end of its diastolic period.

In some embodiments of the invention, the diastolic portion of the heart cycle is cut short, after most of the filling with blood has occurred. As a result of shortening, or trimming, the diastolic portion, a higher heart rate (HR) occurs, and the total volume of blood circulated (cardiac output, or CO) increases. The tradeoff between cutting short the diastolic portion and increasing the heart rate optimizes CO. In short, less time is allowed for waiting for the LV to fill and a higher HR are used to increase CO, rather than more time for filling, leading to a lower HR.

A number of techniques are described which enable sensing when most of the filling with blood has occurred.

The term Diastole Trimmer (Drimmer) is used throughout the present specification and claims for apparatus which acts in accordance with an embodiment of the present invention to shorten diastole. The present invention includes method embodiments which use Diastole Trimming.

According to an aspect of some embodiments of the present invention there is provided apparatus for diastole trimming including a controller for producing a diastole ending signal, and one or more leads connected to the controller, for carrying the signal to lead connections to a heart, characterized by the controller detecting when a left ventricle (LV), and producing the diastole ending signal such that the diastole duration is trimmed.

According to some embodiments of the invention, an amount by which the diastole duration is trimmed is a fixed portion of a threshold value substantially equal to an untrimmed diastole duration.

According to some embodiments of the invention, the controller is configured to send the diastole ending signal via a plurality of the one or more leads.

According to some embodiments of the invention, the controller is configured to produce a diastole ending signal by activating a left atrial contraction.

According to some embodiments of the invention, further including one or more physiological sensors connected to the controller and configured to provide input signals to the controller.

According to some embodiments of the invention, an amount by which the diastole duration is trimmed is based, at least in part, on the input signals.

According to some embodiments of the invention, an amount by which the diastole duration is trimmed is based, at least in part, on the controller analyzing the input signals and determining when a left ventricle of the heart is mostly full.

According to some embodiments of the invention, the decision rules are based, at least in part, on a table which includes values of velocity of ventricular motion and heart rate, and a corresponding time delay from last electric activation to production of the diastole ending signal.

According to some embodiments of the invention, the controller is configured to refrain from producing the diastole ending signal, based, at least in part, on the input signals.

According to some embodiments of the invention, an amount by which the diastole duration is trimmed is calculated every single heart cycle. According to some embodiments of the invention, an amount by which the diastole duration is trimmed is calculated once every several heart cycles. According to some embodiments of the invention, the controller is configured to refrain from producing the diastole ending signal once every two or more heart cycles.

According to some embodiments of the invention, the input signals include a signal based, at least in part, on electric activity of the heart.

According to some embodiments of the invention, the input signals include a signal based, at least in part, whether atrial fibrillation is detected.

According to some embodiments of the invention, the input signals include a signal based, at least in part, on how much blood is in the left ventricle compared to a full left ventricle.

According to some embodiments of the invention, further including one or more accelerometers, and in which the input signals include a signal based, at least in part, on acceleration measured at a wall of the heart. According to some embodiments of the invention, the input signals include a signal based, at least in part, on wall tension measured at a wall of the heart. According to some embodiments of the invention, the input signals include a signal based, at least in part, on oxygen saturation. According to some embodiments of the invention, the input signals include a signal based, at least in part, on blood flow rate. According to some embodiments of the invention, the input signals include a signal based, at least in part, on respiratory rate. According to some embodiments of the invention, the input signals include a signal based, at least in part, on measured heart rate. According to some embodiments of the invention, the input signals include a signal based, at least in part, on measured body motion. According to some embodiments of the invention, the input signals include a signal based, at least in part, on a body's posture. According to some embodiments of the invention, the input signals include a signal based, at least in part, on a temperature.

According to some embodiments of the invention, the input signals include a signal based, at least in part, on an input from a unit external to a patient's body.

According to some embodiments of the invention, an input signal and the diastole ending signal share a lead.

According to an aspect of some embodiments of the present invention there is provided apparatus for diastole trimming including a controller for producing a diastole ending signal, and one or more leads connected to the controller, for carrying the diastole ending signal to a heart, characterized by the controller being programmed to produce the diastole ending signal such that the diastole duration is trimmed.

According to an aspect of some embodiments of the present invention there is provided apparatus for diastole trimming including a controller for producing a diastole ending signal, and a connection to a pacemaker, characterized by the controller having decision rules for indicating to the pacemaker when to fire and end the diastole.

According to an aspect of some embodiments of the present invention there is provided a method of programming a pacemaker characterized by increasing cardiac output by trimming duration of diastole.

According to some embodiments of the invention, further including collecting data from one or more physiological sensors, and in which an amount by which the diastole duration is trimmed is based, at least in part, on analyzing the input signals and determining when a left ventricle of the heart is mostly full.

According to some embodiments of the invention, further including trimming the duration of the diastole based, at least in part, on a table which includes values of velocity of ventricular motion and heart rate, and a corresponding time delay from a last electric activation to production of a diastole ending signal.

According to an aspect of some embodiments of the present invention there is provided a method for increasing cardiac output including producing a signal to trim diastole duration, thereby increasing heart rate (HR) and increasing a product of stroke volume (SV) times HR.

According to some embodiments of the invention, an amount by which the diastole duration is trimmed is a fixed portion of an untrimmed diastole duration as measured by a medical professional.

According to some embodiments of the invention, further including implanting a diastole trimming device in a patient's body.

According to some embodiments of the invention, further including sensing one or more physiological parameters related to a body, and providing the one or more physiological parameters to a controller in the diastole trimming device.

According to some embodiments of the invention, an amount by which the diastole duration is trimmed is based, at least in part, on the input signals.

According to some embodiments of the invention, an amount by which the diastole duration is trimmed is based, at least in part, on a table which includes values of velocity of ventricular motion and heart rate, and a corresponding amount by which the diastole duration is to be trimmed.

According to some embodiments of the invention, the controller is configured to refrain from trimming the diastole, based, at least in part, on the input signals.

According to some embodiments of the invention, an amount by which the diastole duration is trimmed is calculated every single heart cycle. According to some embodiments of the invention, an amount by which the diastole duration is trimmed is calculated once every several heart cycles. According to some embodiments of the invention, the controller is configured to refrain from trimming the diastole once every two or more heart cycles. According to some embodiments of the invention, the input signals include a signal based, at least in part, on electric activity of the heart.

According to some embodiments of the invention, the input signals include a signal based, at least in part, on how much blood is in the left ventricle compared to a full left ventricle.

According to some embodiments of the invention, the input signals include a signal based, at least in part, on acceleration measured at a wall of the heart. According to some embodiments of the invention, the input signals include a signal based, at least in part, on wall tension measured at a wall of the heart. According to some embodiments of the invention, the input signals include a signal based, at least in part, on oxygen saturation. According to some embodiments of the invention, the input signals include a signal based, at least in part, on oxygen consumption. According to some embodiments of the invention, the input signals include a signal based, at least in part, on blood flow rate. According to some embodiments of the invention, the input signals include a signal based, at least in part, on respiratory rate. According to some embodiments of the invention, the input signals include a signal based, at least in part, on measured heart rate. According to some embodiments of the invention, the input signals include a signal based, at least in part, on measured body motion. According to some embodiments of the invention, the input signals include a signal based, at least in part, on a body's posture. According to some embodiments of the invention, the input signals include a signal based, at least in part, on a temperature.

According to some embodiments of the invention, the input signals include a signal based, at least in part, on an input from a unit external to a patient's body.

According to some embodiments of the invention, an input signal and the diastole ending signal share a lead.

According to an aspect of some embodiments of the present invention there is provided a method of treating diastolic heart failure including increasing cardiac output using the above-mentioned methods.

According to an aspect of some embodiments of the present invention there is provided a method of diagnosing diastolic heart failure including using the above-mentioned apparatus.

According to an aspect of some embodiments of the present invention there is provided a method of evaluating suitability of a patient to cardiac output (CO) enhancement by diastole trimming including obtaining a graph belonging to one of a group containing: LV volume over time, myocardial wall velocity over time, and myocardial wall acceleration over time, and assessing whether decreasing diastole time will increase CO.

According to an aspect of some embodiments of the present invention there is provided a method of treating pulmonary edema including increasing cardiac output using the above-mentioned methods.

According to an aspect of some embodiments of the present invention there is provided a method of treating atrial fibrillation including detecting ventricular activation, measuring a time between last two ventricular activations, Tlast, measuring a time to end of a rapid filling phase of LV, Trf, if Trf/Tlast is smaller than threshold, producing a diastole ending signal.

According to an aspect of some embodiments of the present invention there is provided a method of treating bradycardia including detecting ventricular activation, measuring a time between last two ventricular activations, Tlast, measuring a time to end of a rapid filling phase of LV, Trf, if Trf/Tlast is smaller than threshold, producing a diastole ending signal.

According to an aspect of some embodiments of the present invention there is provided a method for minimizing diastolic myocardial wall stress including producing an electric signal to decrease a duration of a diastole.

According to some embodiments of the invention, further including measuring myocardial wall stress at least during the diastole, and producing the electric signal at a time based, at least in part, on the measured myocardial wall stress.

According to an aspect of some embodiments of the present invention there is provided a method for synchronizing computed tomography angiography (CTA) scanning with heart movement, including sensing a physiological parameter of the heart, computing elapsed time within a heart cycle based, at least in part, on a value of the parameter, and providing the elapsed time to a CTA system.

According to some embodiments of the invention, the sensing, the computing, and the providing, are performed by an implanted apparatus.

According to an aspect of some embodiments of the present invention there is provided a method for synchronizing computed tomography angiography (CTA) scanning with heart movement, including sensing a physiological parameter of the heart, computing elapsed time within a heart cycle based, at least in part, on a value of the parameter, and providing a synchronizing signal to a CTA system each instance of a specific time within the heart cycle.

According to some embodiments of the invention, the sensing, the computing, and the providing, are performed by an implanted apparatus.

According to an aspect of some embodiments of the present invention there is provided a method for diagnosing a change in blood supply to a cardiac wall including making a first recording of one or more physiological parameters during LV filling cycle at a first time, making a second recording of the same one or more physiological parameters during LV filling cycle at a second, later time, comparing the first recording to the second recording, and determining whether differences between the first recording and the second recording indicate a change in the blood supply to the cardiac wall.

According to some embodiments of the invention, the making of the first recording includes calculating a mean and a standard deviation of the physiological parameters during the first time, the making of the second recording includes calculating a mean and a standard deviation of the physiological parameters during the second time, and the determining includes determining whether differences between the first mean and the second mean are significant.

According to an aspect of some embodiments of the present invention there is provided a method for tracking cardiac revascularization including making a first recording of one or more physiological parameters during LV filling cycle soon after cardiac revascularization, making a second recording of the same one or more physiological parameters during LV filling cycle later after cardiac revascularization, comparing the first recording to the second recording, and determining whether differences between the first recording and the second recording indicate a change in the efficacy of the cardiac revascularization.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings and images. With specific reference now to the drawings and image in detail, it is stressed that the particulars shown are by way of example and for purposes of illustrative discussion of embodiments of the invention. In this regard, the description taken with the drawings and images makes apparent to those skilled in the art how embodiments of the invention may be practiced.

In the drawings:

FIG. 1A is a simplified block diagram illustration of a Drimmer, constructed according to an example embodiment of the present invention;

FIG. 1B is a simplified block diagram illustration of an example embodiment of the invention;

FIG. 1C is a simplified block diagram illustration of yet another example embodiment of the invention;

FIG. 2 is a simplified illustration of a lead and a method of fixing the lead to a heart wall 301, according to an example embodiment of the present invention;

FIG. 3A is a graph produced by curve fitting Doppler measurements of myocardial tissue velocity, reconstructed from echocardiographic data;

FIG. 3B is a graph of acceleration-deceleration (A-D) of tissue of the left ventricle (LV) produced by time derivation of the graph of FIG. 3A;

FIG. 3C is a simplified qualitative graph of left ventricle volume over a time of a diastole;

FIG. 3D is a simplified graph illustrating effects of LV diastolic failure caused by decreased ventricular compliance on LV pressure-volume loop;

FIG. 3E depicts images of Doppler trans-mitral flow velocity in diastolic dysfunction grade I and grade III;

FIG. 3F depicts graphs of percentage of LV volume as a function of time of diastole;

FIG. 3G depicts graphs comparing LV filling patterns in a normal heart and in DHF patients;

FIG. 3H includes a first graph which depicts LV volume over time in a patient with DHF, and a second graph which depicts LV volume over time in a patient with DHF to which pacing has been applied according to an example embodiment of the present invention; and

FIG. 4 is a simplified flow chart illustration of a mode of operation of an example embodiment of the present invention.

### DESCRIPTION OF SPECIFIC EMBODIMENTS OF THE INVENTION

The present invention, in some embodiments thereof, relates to a method and apparatus for optimizing cardiac performance by controlling time of left ventricular filling, and, more particularly, but not exclusively, to the treatment of heart failure, to apparatuses and methods for treatment of diastolic heart failure, to improving right ventricle output, to preventing backward heart failure and minimizing diastolic myocardial wall stress, in patients with diastolic heart failure. The invention may also be used in other clinical contexts as described below.

Before explaining at least one embodiment of the invention in detail, it is to be understood that the invention is not necessarily limited in its application to the details of construction and the arrangement of the components and/or methods set forth in the following description and/or illustrated in the drawings and/or the examples. The invention is capable of other embodiments or of being practiced or carried out in various ways.

In some embodiments of the invention, a “real-time dialog” between medical treatment and a degree of diastolic dysfunction (DD) or pattern of LV filling is offered.

The present invention, in some embodiments thereof, provides a treatment for DHF, including apparatuses and methods.

Embodiments of the present invention may optionally be used on their own, or as a complement for prior art drug therapy.

The present invention, in some embodiments thereof, optionally produces a “real-time dialog”, or synergy between current drug therapy, including HR slowing drugs and patient's varying pattern of LV filling. It is believed that this synergy will improve the patient's quality of life, and prevent hospitalizations due to acute decompensation of DHF.

The present invention, in some embodiments thereof, measures, optionally at real-time, mechanical characteristics of LV filling, which provide information regarding the pattern of LV filling. The pattern of LV filling indicate whether “most” of LV filling occurred early in diastole (as in DD grade II and III) or late in diastole (as in DD grade I). “Most” of LV filling is optionally determined by interaction with a medical professional. In case where most of LV filling occurred early in diastole, the latter portion of LV filling is optionally sacrificed by activating the heart artificially, optionally by pacing. Stroke volume (SV) is compromised, and HR is increased. The trade off results in a greater CO for the patient. The product SVxHR during intervention (artificial pacing) is controlled to be greater than SVxHR pre-intervention.

The present invention, in some embodiments thereof, provides a way to increase CO even in a patient receiving HR slowing drugs such as beta-blockers.

Further, the present invention, in some embodiments thereof, is designed to operate when patient is anticipating an increase in demand for CO, as during exercise. When patient is at rest and requirement of CO is not increased, this invention will not intervene. Optionally or alternatively, when patient's unassisted HR is greater than a predetermined HR threshold, the invention will not intervene.

The present invention, in some embodiments thereof, uses several physiological inputs from the myocardium to determine physiological parameters, such as, for example, real-time LV filling pattern; a patient's intrinsic HR; and a patient's level of activity (rest or exertion), to result in a decision whether to intervene by artificial pacing of the heart, thereby increasing patient's CO.

Some insights as to some aspects of working of the heart are now described.

Diastolic parameters vary with time as a result of intrinsic and extrinsic factors such as exertion, ischemia, increased afterload (blood pressure), increased preload (left atrial (LA) filling pressure), stress, etc. To emphasize, diastolic parameters are not constants measured in an echocardiographic session, rather these parameters vary.

Diastolic parameters vary spatially in ventricular walls (due to past infarcts, or processes that infiltrate the myocardium).

Decreasing HR, thereby providing longer time for LV filling, is not invariably useful for increasing LV filling and cardiac output (CO). On the contrary, if most of LV filling occurs early in diastole, providing more time for diastole and LV filling may be inefficient. Moreover, prolonging LV filling time will increase LV diastolic wall stress which on the long term may stimulate undesired remodeling processes, while decreasing LV filling time will decrease LV diastolic wall stress.

The present invention, in some embodiments thereof, measures diastolic parameters from different sites of the heart, results in data regarding LV filling. Additional activity and/or metabolic data provide a framework for decision making, whether to pace the heart or not.

The present invention, in some embodiments thereof, provides a patient specific pacing regimen which decreases diastolic LV wall stress and improves CO.

A model of some aspects of how the heart works is now described, which model can be used to understand how to modify embodiments of the invention which are described below.

A distinction between systolic HF (impaired contraction of the heart) and DHF (impaired relaxation, distention and filling of the heart with blood) is important because treatment differs.

The prior art standard of care for DHF, using HR slowing drugs, attempts to increase CO, which is a product of HR and stroke volume, by augmenting the filling of the LV. However, this line of treatment has its limitations. It is uniformly administered to patients regardless of a grade of LV diastolic dysfunction, typically as measured by echocardiography.

It is noted that the grade of LV diastolic dysfunction (DD) determines the pattern of LV filling. For example, in grade I DD a greater portion of the volume filling the LV is obtained during atrial contraction (late in diastole), whereas in grade II and III DD most of LV filling is obtained during the rapid (early) filling phase. The contribution of prior art prolonging of the diastole may differ in the various grades of DD. Specifically, prolonging the diastole may be useful as long as the volume entering the LV late in diastole is significant. If the late diastolic LV filling is small, then prolonging the diastole, by reducing HR, will reduce CO more than increase it.

The prior art standard of care is administered to patients regardless of the varying nature of DD, despite that it is known that the grade of DD is affected by daily stresses such as blood pressure (afterload), volume of blood (preload), and ischemia, as described by Nishimura et al.

Moreover, by prolonging the diastole (with heart rate slowing drugs), and increasing end diastolic LV volume, prior art drug therapy increases end diastolic LV wall stress. Increasing end diastolic LV wall stress elicits stretch induced processes which may exacerbate LV remodeling. For example: increased wall stress releases angiotensin II (AII) from the myocardium. All couples with its sarcolemmal receptor, activating the Gq protein. The Gq protein activates a series of processes which result in the activation of the enzyme complex mitogen-activated protein kinase. The latter enzyme complex is associated with degenerative and adverse processes for the myocyte, such as apoptosis (programmed cell death) and HF. The above process is described in Braunwald 2008; p. 536.

In addition, increasing LV wall stress increases the myocardial consumption of oxygen. This increase must be met by an increase in oxygen supply.

It is thus concluded that current treatment of DHF is not necessarily effective, it was not shown to change the course of DHF, and it did not improve patient survival over the last 25 years (see Das et al., Owan et al., Braunwald 2008; p. 654).

In some embodiments, the Drimmer is an implantable medical device (IMD), however, in some embodiments the Drimmer is a temporarily inserted device.

Some Embodiments Use a Standard Pacemaker

In some embodiments a standard pacemaker is used to pace the heart according to the physiological understandings taught by some embodiments of the present invention.

Programming a standard pacemaker to include the above physiologic understandings to result in an increased CO, optionally takes the following form.

A rule of thumb is optionally used to support an average pace modification using the pacemaker.

For example, the rule of thumb assumes that diastole lasts ⅔ of a heart cycle. By measuring electric activity of the heart cycle, duration of diastole is determined. From, by way of a non-limiting example, echocardiographic measurements of LV wall velocity, a fraction of the duration in which rapid filling occurs is deduced. For example, if LV wall velocity is normal, a fraction of rapid filling relative to the diastole duration is 0.7. If LV wall velocity is between Vi and Vj, the fraction of rapid filling relative to the diastole duration is 0.5. Similarly, if LV wall velocity is between Vk and Vm the fraction of rapid filling relative to the diastole duration is 0.3.

A standard pacemaker is optionally programmed to take into account the fraction of rapid LV filling, optionally based on what stage is reached in the cardiac cycle, optionally based on measured electric activity of the heart, and accordingly to produce a pacing rhythm which positively affects the CO.

An example method for using a programmable pacemaker includes the following:

A physician measures filling pattern for a patient, and decides on an initial set of numbers specifying how much to trim the diastole at each heart rate.

After programming the numbers into a pacemaker, the physician again measures filling pattern for a patient, with the new pacemaker program in effect, and optionally decides on a second set of numbers, if changes are needed, and programs the numbers into the pacemaker. In most cases, the initial set of numbers or the second set of numbers are the last.

Optionally the physician may repeat the measuring the deciding on a set of numbers, and the programming.

Some Embodiments Collect and Use Physiologic Data to Determine Pacing

In some embodiments of the invention, the Drimmer uses data from acquired cardiac and/or body parameters to produce a pacing rhythm. By way of a non-limiting example, the data includes diastolic parameters indicating status of LV filling and distention, and/or body activity parameters (for example respiratory rate) indicating body needs for cardiac output, and/or normal heart rate activity (determined by the sinus node). The Drimmer evaluates the LV filling pattern, for example by analyzing acceleration-deceleration (A-D) signals from A-D sensors in the heart, and according to body needs for cardiac output, determines whether it is beneficial to terminate the diastole earlier.

In an example of how the above embodiment operates, the following scenarios are described. A patient at rest has a HR of 60 beats per minute (patient receives a beta blocker (BB) drug), SV is 50 ml, cardiac output is 3000 ml/min. SV in DHF patients is bounded by the values: 50-65 ml, in contrast to healthy individuals which can increase SV to 100 ml during exercise, see Pina et al. The patient exercises and HR increases to 70/min. This increases CO to 3500 ml/min. Had HR increased to 80/min CO would increase to 4000 ml/min, however, the patient is treated with BB and sympathetic stimulation is blunted by drugs. Since increase of HR is bounded and increase in SV is bounded, CO is bounded. The Drimmer provides a way by which CO is increased.

The Drimmer optionally evaluates the function of LV filling. If most of the LV filling has occurred by a defined time condition, for example: the first third of the diastole period, it is appropriate to terminate the diastole by pacing the heart. The term “most” from “most of the LV filling” is optionally a specific fraction, such, by way of example, 75%, 80% or 90% or intermediate or larger percentages of the blood volume entering the LV.

By way of a non-limiting example, let 85% of said volume define a threshold for early termination of diastole. Then, if the fractional volume is 85% of 50 ml, that is 42.5 ml, after ⅓ of diastole, that is 333 msec, the Drimmer terminates the diastole earlier by pacing the heart. Under normal conditions, with no intervention, the diastole would have continued for some 667 msec. However, using the Drimmer, the diastole is terminated, for example after 700 msec from a last R wave, yielding a HR of 85/min. Multiplying SV of 42.5 ml by HR of 85/min yields a CO of 3612 ml/min.

If diastole terminates at 650 msec, HR increases to 92/min and CO increases to 3864 ml/min.

In some embodiments of the invention the increase of the product SV*HR is optionally processed in real time by the Drimmer.

In some embodiments of the invention the increase of the product SV*HR is optionally processed once every two, or three, or more heartbeats by the Drimmer, and the Drimmer issues changes in heart rate once every two, or three, or more heartbeats.

In some embodiments of the invention, the LV filling pattern is the main datum which determines the pacing.

In some embodiments of the invention, body needs for CO affect the intervention, optionally in addition to or instead of the LV filling pattern.

By way of a non-limiting example if the patient's HR is 120/min, even if due to arrhythmia, optionally, the Drimmer does not intervene in pacing. If patient's LV filling shows 85% of filling within the first third of diastole, and activity parameters do not exceed a set activity threshold, then optionally the Drimmer does not intervene.

In some embodiments of the invention predetermined sets of body parameters are used to determine when to inactivate pacing by the Drimmer. By way of a non-limiting example, when HR>HRth (where HRth is a threshold heart rate suitable for the patient at hand, optionally as determined by a medical professional) and one or more body activity parameters are less than a threshold of the body activity parameters. Examples of body activity parameters include respiratory rate.

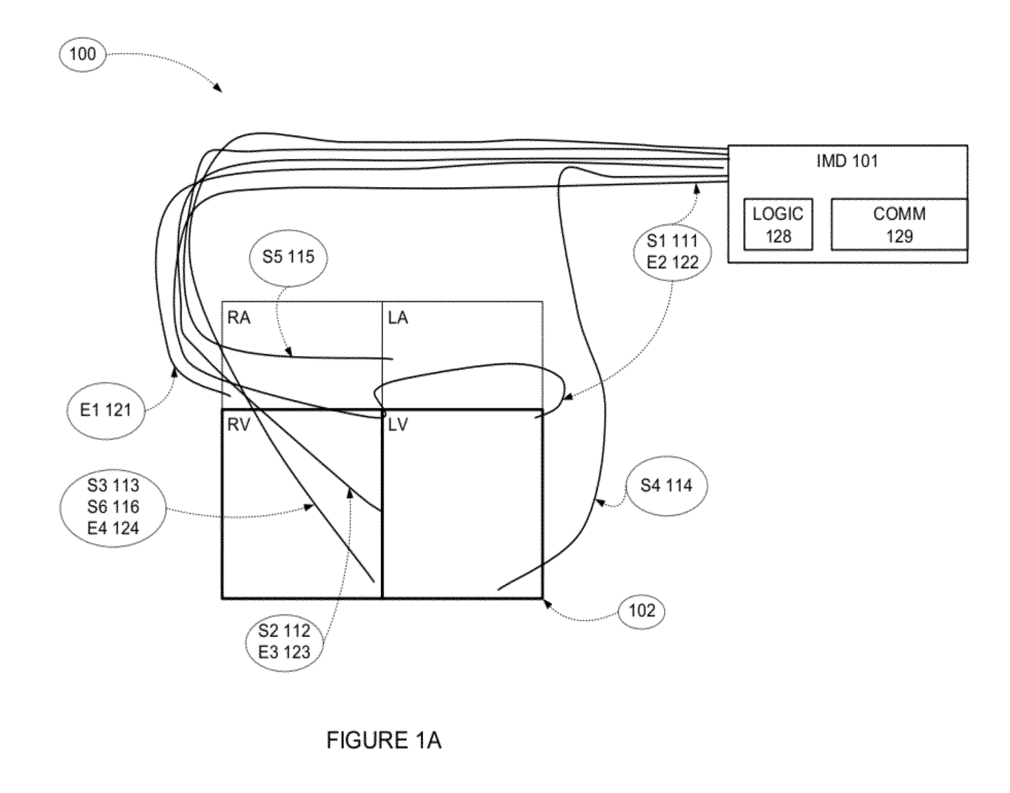

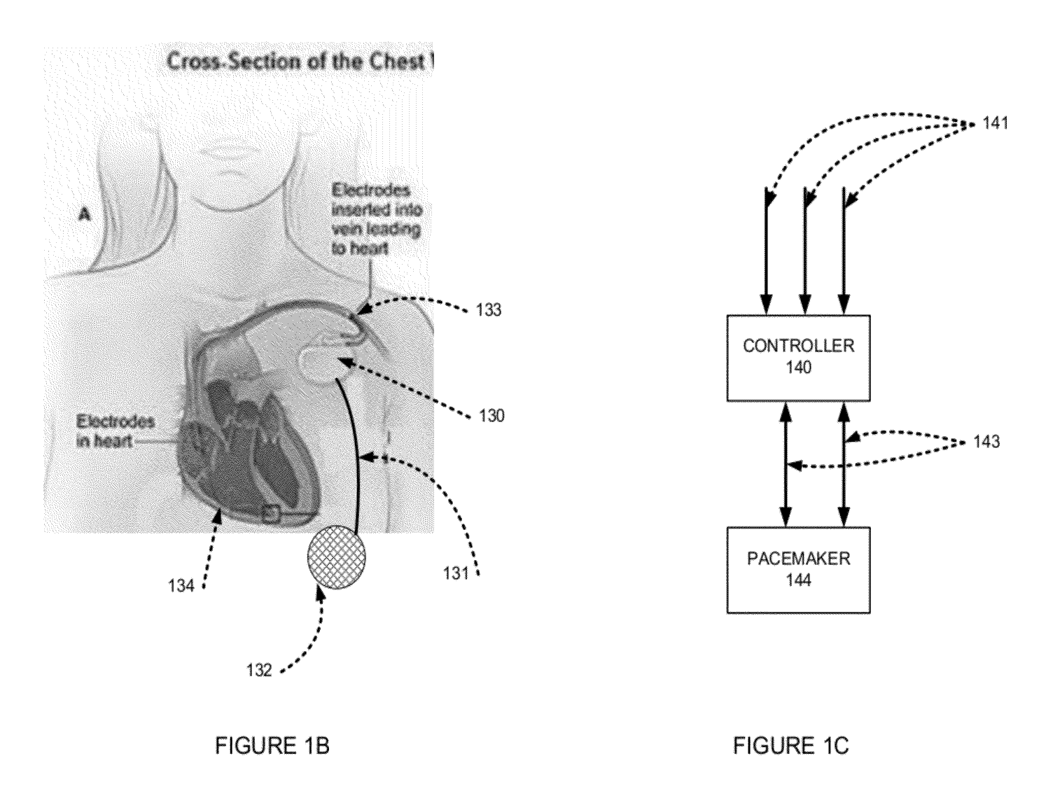

In some embodiments of the invention, the Drimmer optionally acquires, analyzes, and processes data, however if conditions for intervention are not met, an intervening, efferent arm (see FIG. 1A, described below) is not activated.

The efferent arm provides electric stimulation from the Drimmer, which can terminate a diastole earlier.

In some embodiments of the invention, furthermore, the Drimmer does not produce a pacing signal according to some cardiac periodicity or rhythm, such as, by way of a non-limiting example, every n-th heart beat (n=1, 2, 3, . . . ).

In some embodiments of the invention, The Drimmer assists operation of the heart in a sense that only when a set of one or more conditions are met the Drimmer paces the heart. The regular activation of the heart is not disrupted (by pacing) unless conditions are met.

In some embodiments of the invention the Drimmer does not operate as an implantable defibrillator.

In some embodiments of the invention the Drimmer can house a defibrillator feature.

In some embodiments of the invention the Drimmer can hold other features of pacemakers as known in the art, such as anti-tachycardia pacing.

In some embodiments of the invention the Drimmer senses when the LV has completed most of the filling with blood, according to different sensor inputs. The different sensors provide different inputs, such as heart wall acceleration, heart wall velocity, blood flow velocity, oxygen saturation, and more, as will be further described below.

In some embodiments of the invention the Drimmer includes sensors which measure diastolic acceleration-deceleration (A-D) of LV walls in specific locations in the heart.

In some embodiments of the invention the Drimmer includes sensors which measure respiratory motion based on acceleration-deceleration (A-D) of the chest.

In some embodiments of the invention the Drimmer includes sensors which measure physiological variables of metabolism such as CO; temperature; oxygen saturation; and body motion.

It is noted that some measures of activity are known in the art. For example, a step forward in pacemaker function was made in an attempt to mimic nature. Various inputs were utilized to produce a rate-responsive pacemaker, using parameters such as the QT interval, pCO-pCO2 (dissolved oxygen or carbon dioxide levels) in the arterial-venous system, physical activity as determined by an accelerometer, body temperature, ATP levels, adrenaline, etc. Instead of producing a static, predetermined heart rate, or intermittent control, such a pacemaker, a ‘Dynamic Pacemaker’, could compensate for both actual respiratory loading and potentially anticipated respiratory loading. The first dynamic pacemaker was invented by Dr. Anthony Rickards of the National Health Hospital, London, UK, in 1982.

In some embodiments of the invention the Drimmer includes sensors which measure physiological variables of activity, such as, by way of a non-limiting example, posture (supine, upright), HR, and walking.

Reference is now made to FIG. 1A, which is a simplified block diagram illustration of a Drimmer 100, constructed according to an example embodiment of the present invention.

FIG. 1A depicts a heart 102, and the Drimmer 100, which includes an IMD 101, and leads connecting the IMD 101 and the heart 102.

Optionally, the IMD 101 is located under the skin of the chest, in a “pocket”, similarly to a pacemaker. It is noted that the invention is not limited to a form of an IMD, the invention can be an extra-corporal apparatus that is inserted temporarily.

The IMD 101 optionally includes features unique to the present invention, which will be described below, and optionally additional features known in the art such as, by way of a non-limiting example: anti-tachycardia pacing, pacing in cases of atrial fibrillation or bradycardia, defibrillation, and resynchronization therapy.

The IMD 101 optionally interacts with the heart 102 via afferent and efferent leads. The afferent leads optionally carry signals from the heart to the IMD 101, the efferent leads optionally export signals from the IMD 101 to the heart 102. Each lead is composed of at least one electric conductor. It is possible that a lead will perform both afferent and efferent tasks, for example a lead S1 located within the coronary sinus (CS), may optionally bring the signal of acceleration-deceleration (A-D) from the CS to the IMD, and it may also optionally serve as a pacing electrode at that site.

Afferent leads S1 111, S2 112, S3 113, S4 114, S5 115, and S6 116 carry signals from the heart 102 to the IMD 101, and efferent leads E1 121, E2 122, E3 123, and E4 124 carry signals from the IMD 101 to the heart 102.

Signals acquired from the heart 102 are processed in the IMD 101 to optionally result in signals exported to the heart 102. Several sensors are positioned in unique locations in the heart (S1 111, S2 112, S3 113, S4 114, S5 115, and S6 116). The locations for placing the sensors may be uniquely determined for each patient, optionally using echocardiography and/or A-D measurements.

The IMD 101 optionally receives inputs regarding the state of the patient, for example, upright vs. lying position, tachypnea, walking motion, and so on.

In some embodiments of the invention, the IMD 101 includes a logic module 128, for implementing optional processing and/or optional storage.

In some embodiments of the invention, the IMD 101 includes a communications module 129, for implementing optional communications with external devices. Such communications may be, for example, to a pacemaker (details of which are provided below), or to a CT system (details of which are provided below).

In some embodiments, the IMD 101 is optionally activated under specific conditions and deactivated under another set of conditions. For example, the IMD 101 may be deactivated at a heart rate >HRth (e.g. 100 BPM), or when a CO is sensed to be >COth (some value of threshold of CO, e.g. 6 liters/min).

It is noted that in case of some inputs to the IMD 101, processing may occur over a period of more than one heartbeat. For example, since CO is a variable which typically changes over several heartbeat cycles, processing is allowed to be performed over N cycles prior to optionally activating a mode of pacing. An initial value for the number N is optionally determined by a physician, and optionally changes according to different physiological states, optionally dependent on signals from sensors.

Sensors—Afferent Leads in the Example of FIG. 1a:

The sensor 51 111 is located within the CS. The sensor 51 111 is optionally positioned in the vein at a location best demonstrating diastolic motion of the lateral LV wall.

The sensor S2 112 is located at a right side of the inter-ventricular septum. The sensor S2 112 is attached and/or screwed to the muscle. The sensor S2 112 is optionally positioned in an area best demonstrating the diastolic motion of the septum.

The sensor S3 113 is located at the right apex of the right ventricle (RV). The sensor S3 113 is attached and/or screwed to the muscle. The sensor S3 113 is optionally positioned in an area best demonstrating the diastolic motion of the apex of RV.

The sensor S4 114 is located at the apex of the LV. The sensor S4 114 is optionally positioned in an area under the skin, above the ribs, outside the rib cage and above the apex of the LV, where the apical thrust is best palpated with the hand (not attached to the heart). Positioning of the sensor S4 114 is optionally performed by producing a tunnel under the skin from the apparatus.

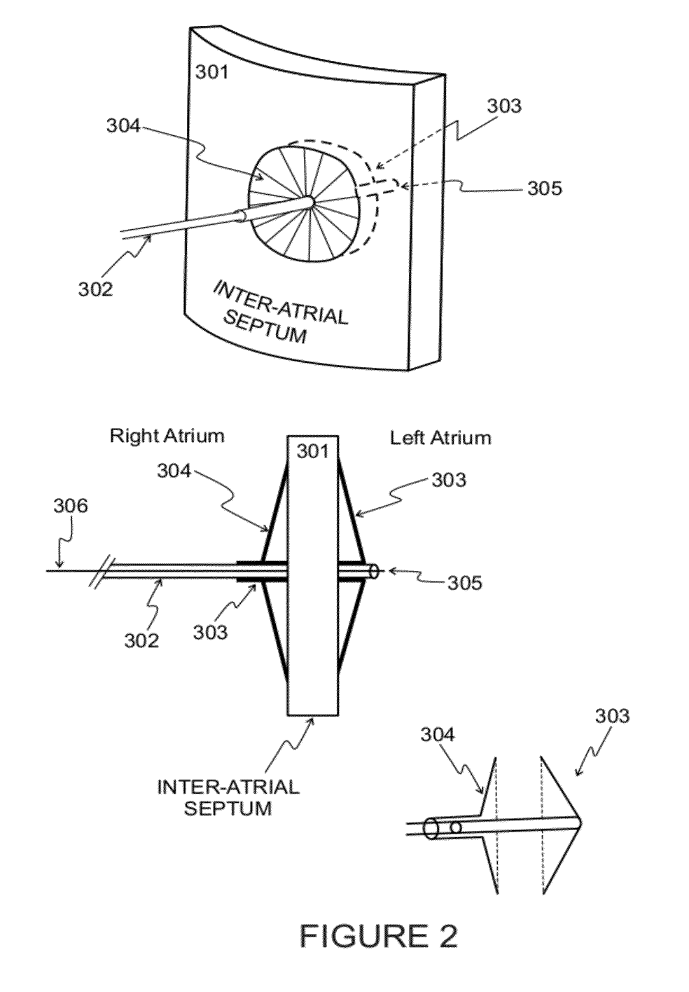

The sensor S5 115 is located in the inter-atrial septum. The inter-atrial septum is penetrated, optionally pierced with a specific catheter to form a tiny hole through which a lead is inserted. The sensor S5 115 is secured to the inter-atrial septum, optionally using a double “umbrella” construction as further detailed with reference to FIG. 2.

The sensor S5 115 measures hemodynamic parameters such as flow or pressure from LA. In one embodiment of the invention, at the tip of sensor S5 115 there is a pressure sensor which indicates whether LA pressure is within a predetermined set of values. An example, for predetermined intervals: <10 mmHg, >10 mmHg and <18 mmHg, >18 mmHg. These values are examples, and specific values may be determined differently for each patient.

The sensor S6 116 measures CO in the RV. Some techniques for measuring CO using an implantable sensor include a hot wire, a thermocouple, or some other technique.

It is noted that while many sensor leads are depicted in FIG. 1A, embodiments of the invention many include no sensors, or one sensor connected to one of the sensor leads, or more than one sensor. FIG. 1A depicts a multi-sensor embodiment, for sake of illustration

Efferent Leads in the Example of FIG. 1A:

The lead E1 121 is a sense-pace electrode in the right atrium (RA). The E1 121 lead senses an atrial and ventricular electrogram from which an R-R interval, the time interval between two successive R wave peaks, is calculated. The lead E1 121 may be used to pace the RA.

The lead E2 122 is a sense-pace electrode positioned in the CS and may be used in bi-ventricular pacing of the LV. The lead E2 122 and the lead S1 111 may optionally be the same lead.

The lead E3 123 is an electrode which optionally alters electric properties of the septum using techniques previously described which are known in the art. The lead E3 123 and the lead S2 112 may optionally be the same lead.

The lead E4 124 is a sense-pace electrode in the apex of the RV. The E4 124 lead senses an electric activity of the ventricle and may be used to pace the heart 102. The lead E4 124 and the lead S3 113 may actually be the same lead.

Some of the leads pace the heart. For example in the right atrium (RA) or right ventricle (RV).

Some of the leads perform a role of modifying diastolic properties of the heart, as will be detailed further below, in a section headed “Decreasing diastolic LV wall stress”.

In some embodiments of the invention the raw signals and/or their measures are optionally stored for later use.

Optionally, after comparing measures with threshold values, and/or optionally making calculations based on the inputs, an optional decision process is performed for deciding output of the IMD 101.

The output may be a pacing signal for the RA, or for the RV, or for both the RV and LV.

The output may also be a conditioning signal which modifies the diastolic properties of a LV wall.

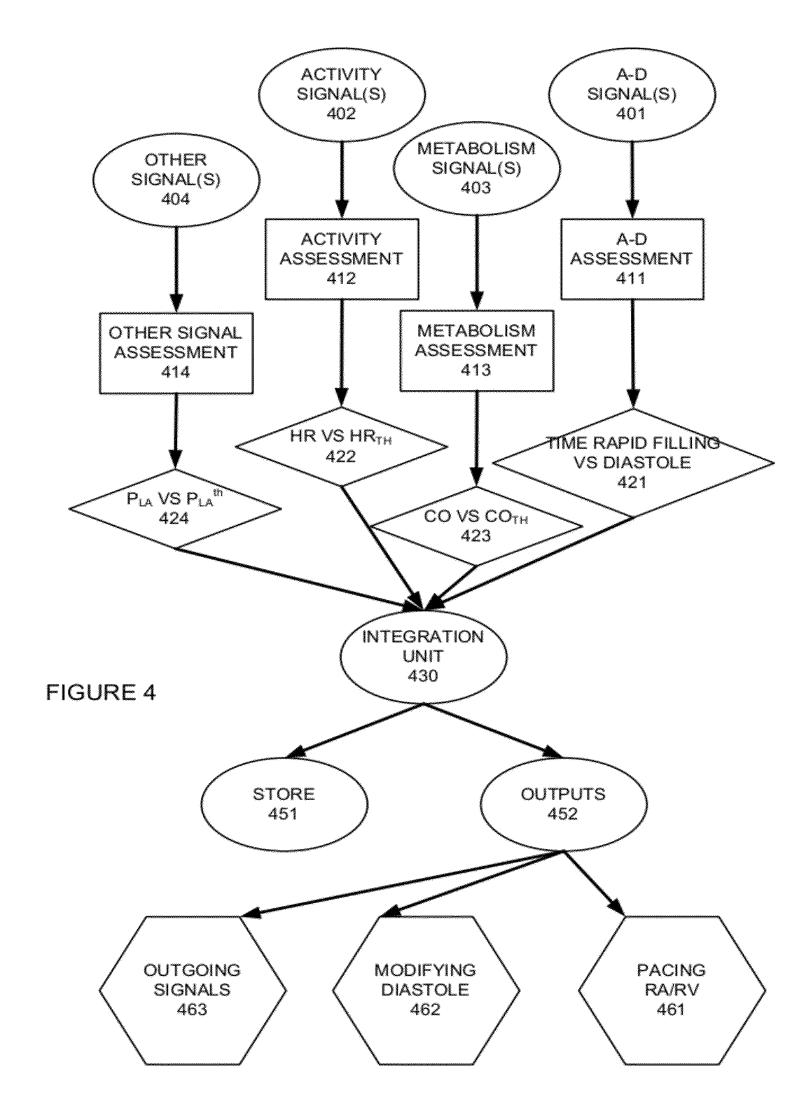

The IMD 101 optionally includes a processing unit which integrates the inputs from the sensors to effect an output, which is optionally a pacing stimulus that terminates diastole. The processing is further described below, with reference to FIG. 4.

The IMD 101 optionally includes a processing unit which stores relevant information, optionally for exporting it to a monitoring or an interrogating system.

The IMD 101 optionally includes a processing unit which optionally sends relevant information for external monitoring and/or analysis, and/or storage.

The output of the IMD 101 includes output of one or more of the following types:

Pacing the heart—determining an optimal time for terminating diastole in order to optimize CO. It is possible that pacing as taught according to the present invention be provided in addition to pacing forms known in the art.

Modifying diastolic properties of ventricular wall by electric stimulation, optionally as known in the art.

Storing data for further analysis and/or intervention.

Optional placement of the sensor S4 114 in an area under the skin, over the ribs, outside the breast cage, and above the apex of the LV, opens a new possibility, and is used in some embodiments of the invention.

In some embodiments of the invention an A-D sensor is located under the skin and above the ribs, where the apical thrust can be felt strongly. The sensor is connected to a controller similar to the IMD 101. The controller processes input from the sensor, following the pulsating of the heart, this tracking the cardiac cycle, when the heart is moving, when the heart is still. The controller detects the end of the LV fill cycle, which is an instance of stillness, and optionally produces a signal for trimming the diastole, whether by atrial stimulation, by a later ventricular stimulation, or both.

Two types of embodiments are contemplated: apparatus including the apical sensor, connected by a lead or leads to the controller, connected by one or more stimulation leads to the heart; and apparatus including the apical sensor, connected by a lead or leads to the controller, connected by one or more leads to a pacemaker, which stimulates the heart.

In some embodiments of the invention, which include connection to a pacemaker, the pacemaker is configured to accept a signal for inducing cardiac stimulation, whether by atrial stimulation, by a later ventricular stimulation, or both. The controller is optionally configured to be subcutaneous, saving a need to open the rib cage.

In some embodiments of the invention, which include connection to a pacemaker, the pacemaker is configured to provide electric power to the controller via a connecting lead.

In some embodiments of the invention, which include connection to a pacemaker, the pacemaker is configured to accept electric power from the subcutaneous controller via a connecting lead.

Reference is now made to FIG. 1B, which is a simplified block diagram illustration of an example embodiment of the invention.

The example embodiment of FIG. 1B includes a controller 130, connected by a lead 131 to an A-D sensor 132. The A-D sensor 132 is optionally located subcutaneously, substantially at or near to a point of maximum thrust from the heart.

The controller 130 is also connected to one or more electrodes 133, which optionally thread through a vein leading to the heart 134, and are attached to the heart at locations appropriate for excitation.

Location of the controller 130 is similar to location of a subcutaneous pacemaker, that is, where leads can reach from the apically placed sensor 132 and leads can reach into a vein, and from there into the heart 134.

While a subcutaneous pacemaker depends on electric activity of the heart for input, a Drimmer constructed according to the example embodiment of FIG. 1B depends on input from the A-D sensor 132. Where the subcutaneous pacemaker provides stimulation to pace a heart according to present pacemaker methods, the Drimmer of FIG. 1B optionally trims the diastole of the heart cycle, achieving increased CO.

Reference is now made to FIG. 1C, which is a simplified block diagram illustration of yet another example embodiment of the invention.

The example embodiment of FIG. 1C includes a controller 140, having one or more input leads 141 from sensors (not shown), and one or more outputs 143 to a pacemaker 144. The example embodiment of FIG. 1C implements the logic of a Drimmer, using a pacemaker to excite the heart.

The pacemaker 144 optionally includes new functionality enabling the pacemaker 144 to accept a control signal from the controller 140 instructing the pacemaker 144 to excite the heart, optionally at a specific location (atria, ventricles).

In some embodiments of the invention the pacemaker 144 optionally includes a connection for connecting one of the leads 143, to provide the controller 140 with power.

In some embodiments of the invention the controller 140 optionally includes a battery, and the pacemaker 144 optionally includes a connection for connecting one of the leads 143, enabling the controller 140 to provide the pacemaker 144 with power.

Reference is now made to FIG. 2, which is a simplified illustration of a lead 302 and a method of fixing the lead 302 to a heart wall 301, according to an example embodiment of the present invention.

FIG. 2 depicts a device for fixing the lead 302 to the heart wall 301, which includes umbrellas 303 304 to fix the lead 302 and a sensor 305 to the heart wall 301.

In FIG. 2 the heart wall 301 is, by way of a non-limiting example, the inter-atrial septum, and the lead 302 approaches the heart wall 301 via the left atrium.

A guide wire 306 is optionally manipulated through the RA toward the inter-atrial septum, in a catheterization procedure.

Fixing the lead 302 includes making a small defect through the septum, depicted from right to left in FIG. 2, optionally using a catheter which is optionally retracted after making the defect. Over the guide wire 306 the distal part of the umbrella 303 is directed to the left side of the septum. The umbrella 303 is folded to optionally form a small tube, and is optionally covered by retractable tubing.

Once the distal part of the umbrella 303 is in place, the covering tubing is optionally retracted by pulling it back, and the umbrella 303 opens, covering the left side of the septum. The covering tube is then optionally removed, and optionally over the same guide wire 306 the proximal side of the umbrella 304 is manipulated. The proximal side of the umbrella 304 optionally has a diameter just greater than that of the umbrella 303. The umbrella 304 is deployed over the umbrella 303, and scaffolds the septum from both sides. After the umbrella 304 is deployed, and the guide wire 306 is optionally still within it, the lead 302 with a sensor 305 is manipulated over the guide wire 306 to its location.

In the example of FIG. 2 the sensor 305 protrudes a bit into the left atrium.

It is noted that fixing the lead 302 and the sensor 305 to the heart wall 301 may be performed in different ways, Especially, the lead 302 and the sensor 305 may optionally be designed as a single piece, with the distal part opening first and the proximal part opening later.

The sensor 305 may be any one of implantable sensors for picking up data such as described below, especially with reference to FIG. 4.

In some embodiments of the invention the sensor 305 is a single sensor. In some embodiments of the invention multiple sensors 305 are implanted. By way of a non-limiting example, one A-D sensor may be implanted, and is sufficient for some example embodiments of the invention. By way of another non-limiting example, a plurality of A-D sensors are used, for example four sensors: one sensor at the coronary sinus; one sensor at an apex of the right ventricle; one sensor at the septum on the right ventricular side; and one sensor subcutaneously at a point of substantially maximal cardiac impulse.

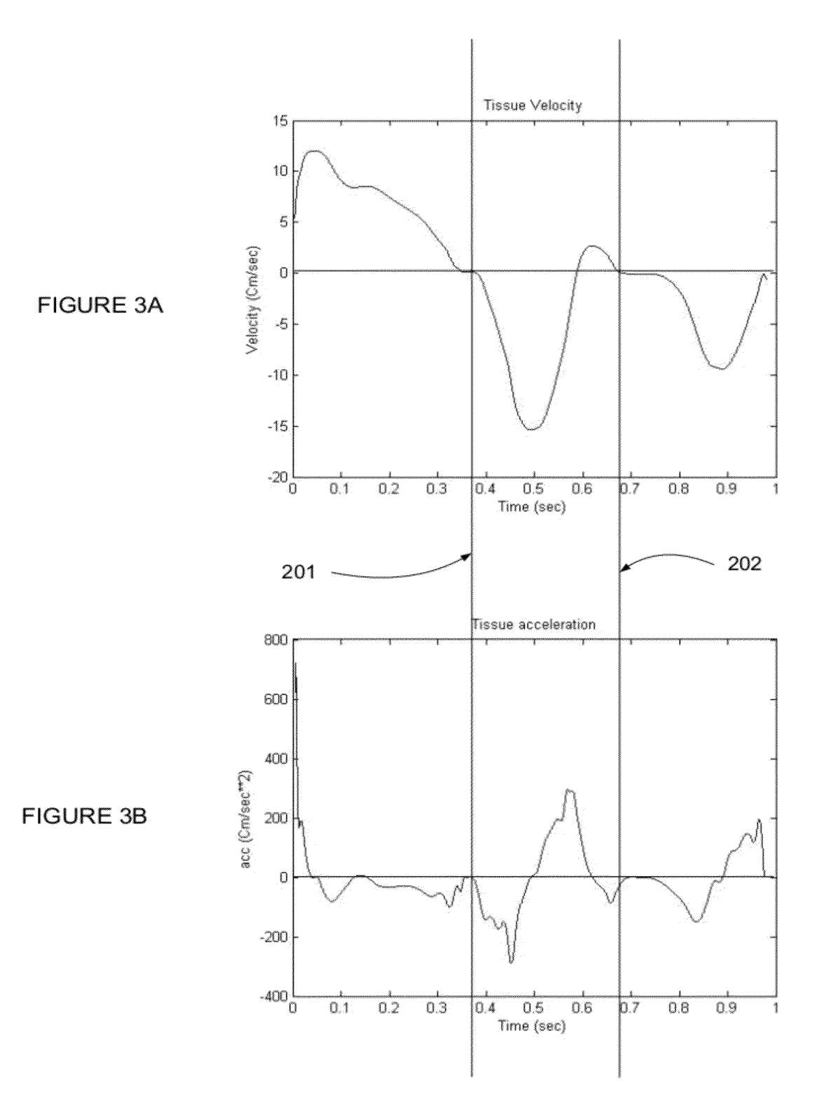

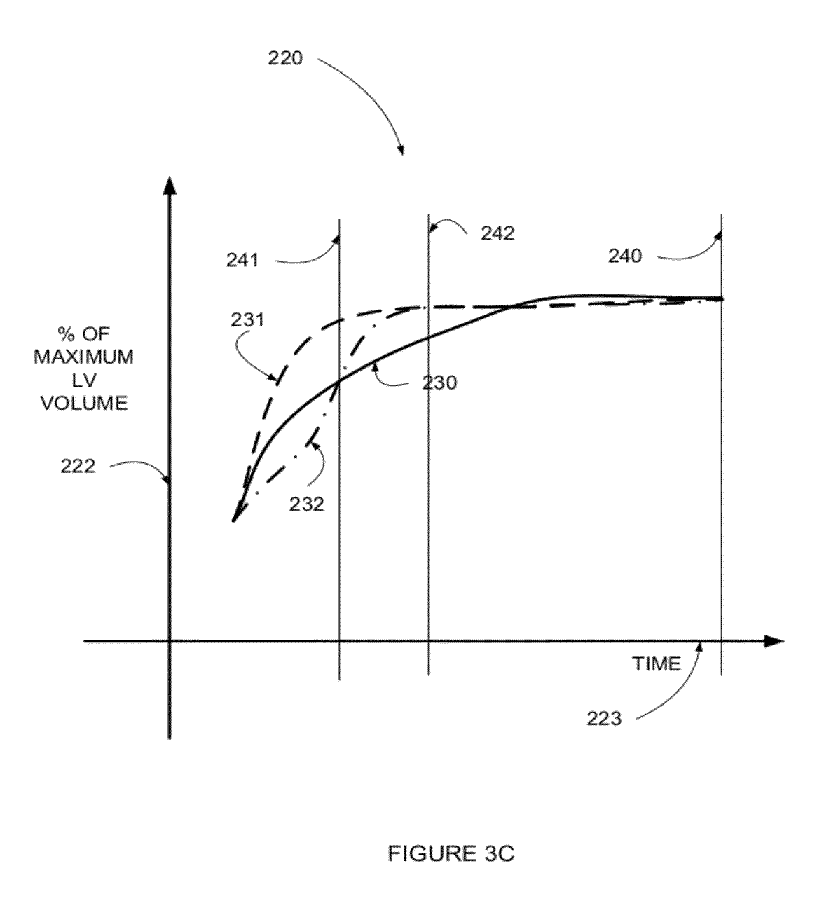

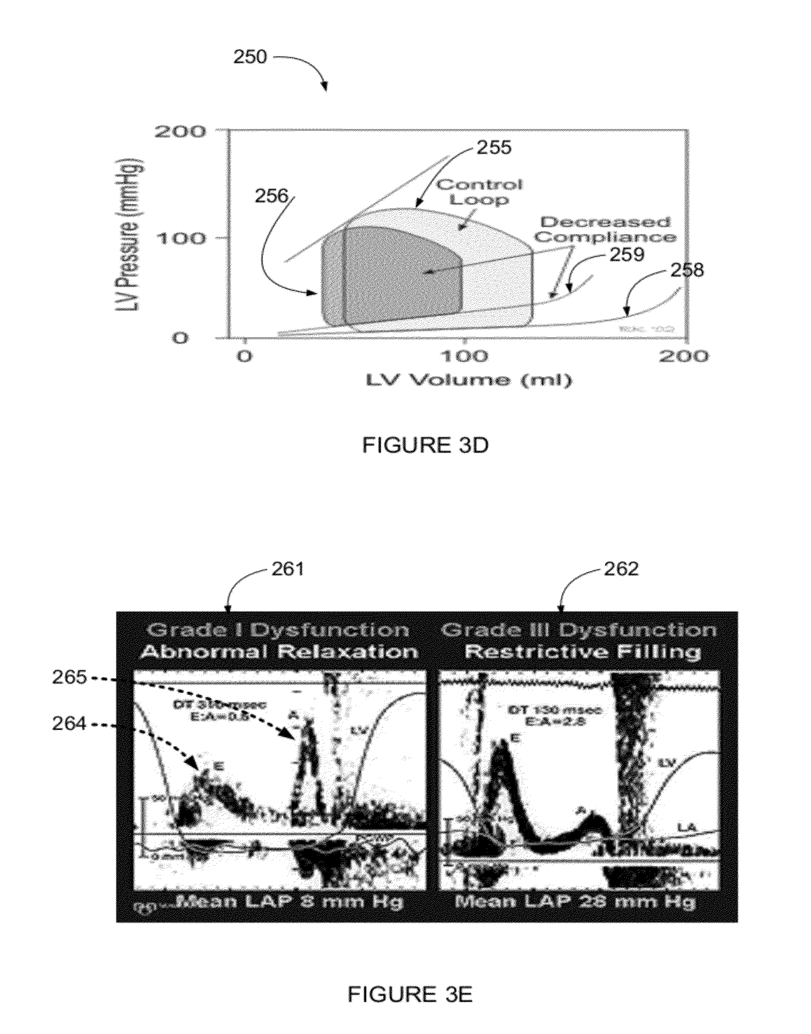

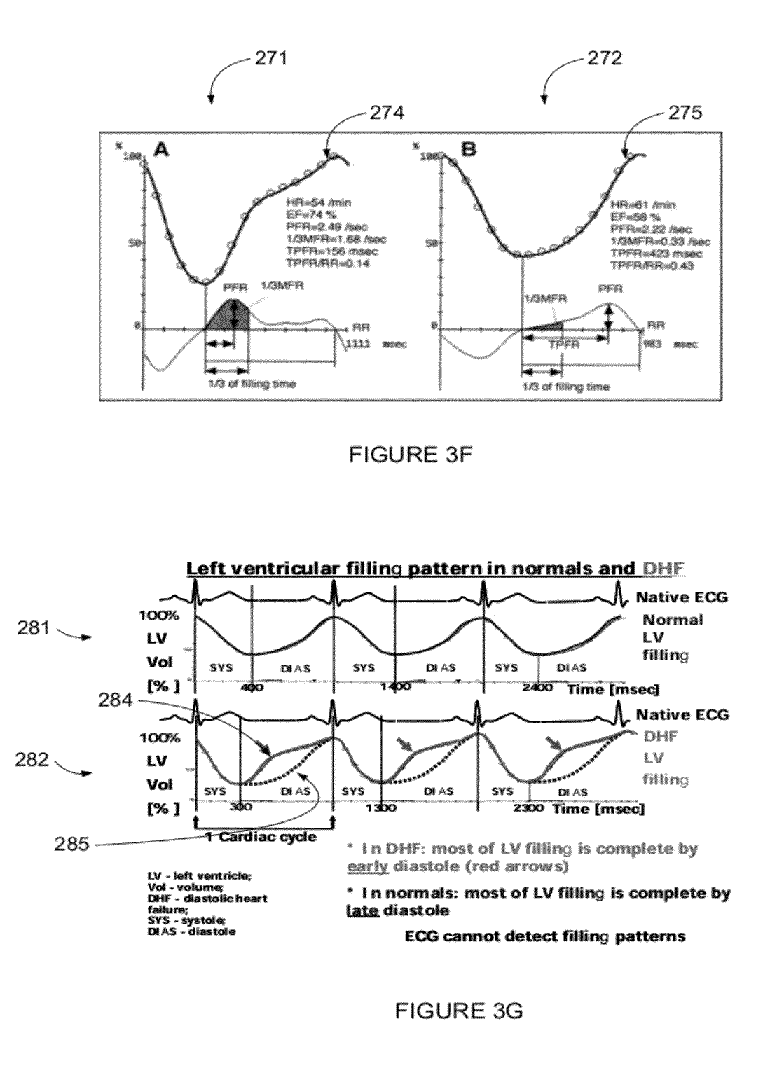

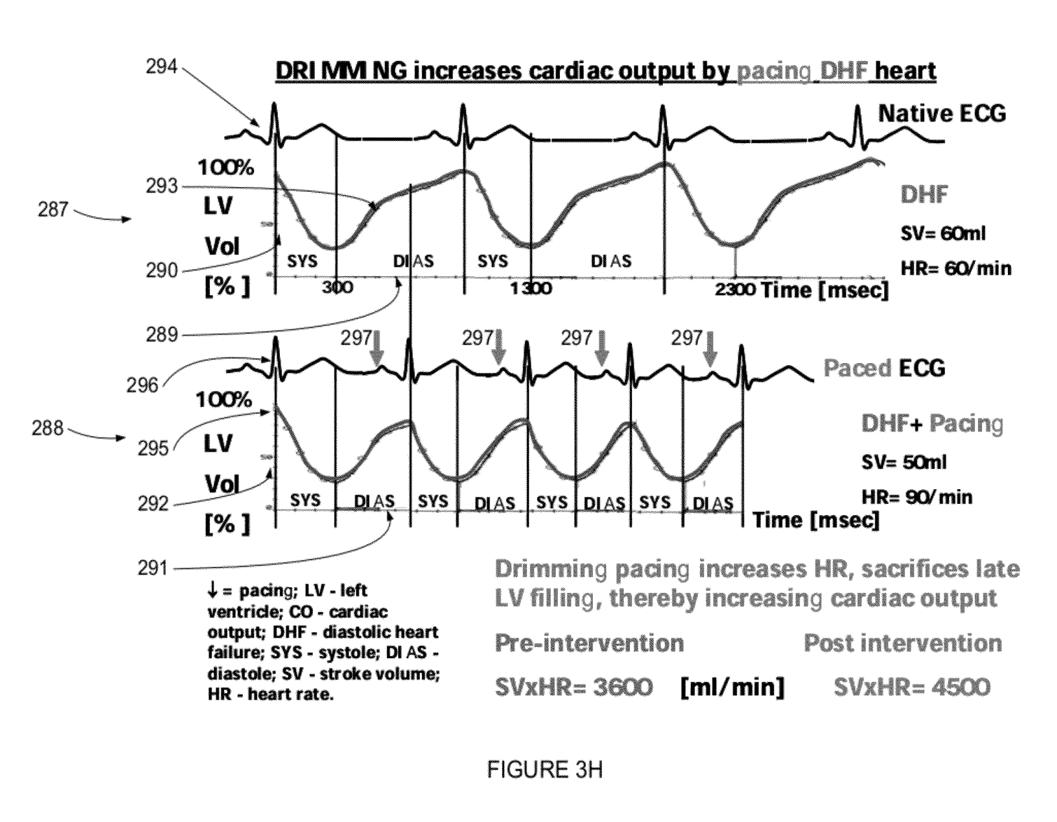

Reference is now made to FIG. 3A, which is a graph produced by curve fitting Doppler measurements of myocardial tissue velocity, reconstructed from echocardiographic data, and to FIG. 3B, which is a graph of acceleration-deceleration (A-D) of tissue of the left ventricle (LV) produced by time derivation of the graph of FIG. 3A.

It is noted that the graph of A-D of tissue of the left ventricle (LV) may also optionally be produced by A-D sensors attached to the LV, in an embodiment of the Drimmer which includes implanted A-D sensors.

The X-axis of both graphs depicts time, 1 second, which represents a single cardiac cycle.

In FIG. 3A, a systolic phase starts at time 0 and ends with a first vertical line 201. Diastole starts with the first vertical line 201 and ends at time 1 sec.

A rapid filling occurs between the first vertical line 201 and a second vertical line 202.

In embodiments of the invention, the time of rapid filling (Trf) is compared with the time of diastole, and a decision is optionally made whether to take actions such as pacing, diastolic property modification, etc.

Some numerical examples are now detailed. Echocardiographic measurements show that LV wall displacement, for example of the septum, is 0.5-1.0 cm. Most of this displacement is obtained within 0.3 sec. Septum velocity is approximately 1.6-3.3 cm/sec. It is estimated that LV septum accelerates from 0 to maximum velocity within 0.1 sec. Therefore, acceleration of the septum is 16-33 cm/sec2.

A commercial over the counter accelerometer having 16 bit resolution and a measurement range of +/−2 g (1 g=9.8 Newton×meter/sec²) can measure: 3×10⁻⁵ g=0.03 cm/sec2. It is noted that the commercial over the counter accelerometers are able to measure the accelerations of LV walls.

Reference is now made to FIG. 3C, which is a simplified qualitative graph 220 of left ventricle volume over a time of a diastole.

The graph of FIG. 3C has an X-axis 223 of qualitative time, without specifying units, and a qualitative Y-axis 222 of percent (%) of a left ventricle (LV) maximum volume.

A first trace 230 depicts a normal expansion of the left ventricle over the time of the diastole. The expansion occurs faster at the beginning of the diastole, and slower later, reaching some maximum volume.

A second trace 231 depicts a problem expansion of the left ventricle over the time of the diastole. The expansion occurs faster at the beginning of the diastole, even faster than a normal expansion, and slower later, reaching substantially close to a maximum volume faster than the normal trace.

A third trace 232 depicts a second problem expansion of the left ventricle over the time of the diastole. The expansion occurs slower at the beginning of the diastole, slower even than a normal expansion, then expands faster, faster even than a normal expansion, reaching substantially close to a maximum volume faster than the normal trace.

A vertical line 240 depicts a normal end of the diastole, a time at which the LV of the normal heart is full.

It is noted that the first trace 230, the second trace 231, and the third trace 232 do not depict additional filling of the left ventricle which occurs at the end of the diastole, and is termed atrial kick. The atrial kick occurs when the atrium contracts, and adds additional blood to the left ventricle. The traces 230 231 232 of FIG. 3C compare left ventricle filling before the atrial kick.

It is also noted that an amount of blood added by the atrial kick to the left ventricle depends on whether or not a patient has atrial fibrillation.

A vertical line 241 depicts a time at which the LV of the heart of the second trace 231 is already mostly full of blood. The volume of the blood in the LV is not as great as in the LV of the normal heart. As already described above, it is advantageous to trim the diastole of the heart of the second trace 231, at the time of the vertical line 241, and gain a greater CO by having a greater HR with a smaller SV. It is noted that the SV will not increase substantially if the diastole duration is allowed to be longer.

A vertical line 242 depicts a time at which the LV of the heart of the third trace 232 is already mostly full of blood. The volume of the blood in the LV is not as great as in the LV of the normal heart or of the heart of the second trace 232. As already described above, it can be advantageous to trim the diastole of the heart of the third trace 232, at the time of the vertical line 242, and gain a greater CO by having a greater HR with a smaller SV. It is noted that the SV will not increase substantially if the diastole duration is allowed to be longer.

Reference is now made to FIG. 4, which is a simplified flow chart illustration of a mode of operation of an example embodiment of the present invention.

FIG. 4 depicts a flow chart including signal acquisition, comparing parameters derived from acquired signals to reference values, sending results of the comparisons into an integration unit. The outputs of the integration unit include: pacing, storage of parameters, and wall stiffness priming.

Ellipses 401 402 403 404 in the upper part of the flow chart depict some examples of raw signals acquired from sensors, optionally in real-time: A-D signal(s) 401, LA pressure (not shown), R-R interval (not shown), activity signal(s) 402, metabolic signal(s) 403 (exercise vs. rest, upright vs. rest position, tachypnea, and temperature), and optionally other signal(s) 404.

In some embodiments of the invention, the raw signals are optionally acquired in real time, that is, approximately once per heartbeat, or several times per heartbeat.

The raw signals 401 402 403 404 are optionally processed by the Drimmer 100 (FIG. 1A) to produce other signals or data parameters 411 412 413 414 which reflect the raw signals, and optionally extract a significance of the raw signals.

Some examples of sensor input to LV filling are now described.

For example, when wall has high acceleration, ventricle is filling, when acceleration substantially lowers, most of the filling is done.

For example, when wall has high velocity, ventricle is filling, when velocity substantially lowers, most of the filling is done.

For example, when blood flow velocity at the ventricle is high, ventricle is filling, when blood flow velocity substantially lowers, most of the filling is done.

Diamonds 421 422 423 424 depict example comparisons of the parameters with threshold values.

By way of a non-limiting example, FIG. 4 depicts a comparison of time of rapid filling vs. diastole 421; HR vs. HR_{TH} 422; CO vs. CO_{TH} 423; and P_{LA} vs. P_{LA}^{TH} 424;

In some embodiments of the invention, threshold values are optionally updated in real time, that is, approximately once per heartbeat, or several times per heartbeat.

An output of the comparisons is optionally another set of data, which enters an integration unit 430.

The integration unit 430 optionally weighs the data, and, according to decision rules, exports a set of one or more outputs.

In some embodiments of the invention the output may be stored 451. The storing optionally includes raw data, and/or results of processing the data, and/or decisions.

In some instances the output of the apparatus optionally takes a form of no action or no pacing action, based, at least in part, on the inputs to the integration unit 430.

In some instances output(s) 452 of the apparatus optionally causes action, such as a pacing regimen for pacing, by way of a non-limiting example, the right atrium RA and/or the right ventricle RV 461; a stimulation to a LV wall that modifies diastolic properties of the LV 462; and/or other outgoing signals 463, including transmitting data to an additional unit, such as external to the body.

In some embodiments of the invention the integration unit 430 maintains, by pacing, a minimum heart rate HRmin.

It is noted that some of the raw signals from the sensors optionally track actions taken by the Drimmer, and optionally measure signals which are relevant to a goal of the integration unit 430, for example CO. These signals are optionally fed back into the apparatus for evaluation whether further intervention is required.

An example method for the integration unit 430 to optionally weigh the data, and apply decisions, is now described:

Within a time period of N seconds (e.g. N=30 seconds) the integration unit 430 acquires signals from body and heart of a typical patient, in a data acquisition phase. For this example, a typical patient is 74 years old, with many years of hypertension causing changes in the left ventricle (LV) known as concentric hypertrophy (homogenous thickening of the walls composing the LV). Such a patient is typically in sinus rhythm, and an ejection fraction per echocardiography is preserved (>50%). During the data acquisition phase optionally no action is taken by the Drimmer. The signals acquired include:

A body activity signal, from example respiratory rate (RespR), measured in units of number of respirations per minute. RespR is averaged over N or more seconds.

A cardiac electrogram acquired from two electrodes in the heart or one electrode within the heart and a conducting surface of a housing unit of the Drimmer apparatus, which serves as a reference electrode. The cardiac electrogram is analyzed, for example by peak signal detection, producing a time interval between consecutive electrical activations (referred to here as RRint). RRintj represents a specific heart cycle j. Ddiasj is a term for a duration of the diastole during heart cycle j, where: Ddiasⱼ typically equals ⅔*RRintj

From sensors measuring ventricular wall motion (for example an accelerometer), the time elapsed between end of systole j and end of rapid filling phase j is obtained (as illustrated in FIG. 3B, the time period between vertical line s201 and 202) and referred to as: RFetj (i.e. rapid filling elapsed time during heart cycle j).

After N seconds of data acquisition, the following exemplary inputs are available: RespR; RRintj; Ddiasj; and RFetj.

Some of the data acquired from heart cycle j is available only after heart cycle j has been completed.

Moreover, t designates real time measured by a timer.

A next phase of operation takes place within the time spanned by heart cycle j+1. All calculations are performed within a short time frame of, for example, 1-2 msec after the last datum is measured, which is very short compared to a cardiac cycle. In this example the last datum measured is RFetj+1. In other words, when RFetj+1 is obtained as explained above, the Drimmer stores this value as t(RFetj+1).

RRintj+1 is assumed to be equal to RRintj. To check this assumption a mean and a standard deviation of RRintj over the last K consecutive cycles (K=integer) are calculated in real time and optionally stored. The standard deviation of RRintj is compared with a reference value. If the standard deviation of RRintj is smaller than a predefined value then the assumption holds, and RRintj+1 is determined safely. If RRintj+1>600 msec then variable go-on1=1, otherwise go-on1=0.

Ddiasj+1 is calculated from RRintj+1 as above.

RFetj+1 is measured in real-time as explained above.

If RespR>RespRth (for example RespRth=15) then go-on2=1;

If RespR>30, B=0.5;

If RespR<30 and 25, B=0.6;

If RespR<25 and 20, B=0.75;

If RespR<20 and 15, B=0.85;

If RespR<15, B=0;

Otherwise go-on2=0.

The variable Aj+1 is calculated where Aj+1=RFetj+1/Ddiasj+1.

If Aj+1<Ath, where Ath is a reference value, which is also a monotonic function of RRintj, such that as RRintj becomes a smaller quantity, Ath also decreases, then a variable tdiflj+1 is calculated.

tdiflj+1=Ddiasj+1−RFetj+1

In words, tdiflj+1 is the time interval (from termination of rapid filling phase j+1 to expected end of diastole j+1) during which a diastole ending signal may be applied.

An additional action is optionally to calculate the time for delivering the diastole ending signal.

If go-on1=1, (condition RRintj+1>600 msec, that is heart rate does not exceed a threshold), and if go-on2=1, (condition RespR is greater than RespRth, this means that patient is active, in contrast to resting) then Tdej+1=tdiflj+1*B, where Tdej+1 is the time that must be added to t(RFetj+1).

When t=t(RFetj+1)+Tdej+1 then a diastole ending signal is fired from the Drimmer.

If go-on1 not equal 1 or go-on2 not equal 1, then, a diastole ending signal is not fired within heart cycle j+1.

End of heart cycle j+1.

In some embodiments of the invention optimizing of the diastolic trimming is performed, based on sensing physiological data which measures cardiac output, directly or indirectly. For example, oxygen saturation is a measure of physiological data which reflect cardiac output. The time by which the diastole is trimmed is varied by the integration unit 430, somewhat up and somewhat down, seeking a maximum to the oxygen saturation. The size of the changes to the time by which the diastole is trimmed may decrease in time, for example starting with a relatively change, and halving the change until n optimum in oxygen saturation is reached.

Some Exemplary Modes of Action of Some Embodiments of the Invention:

Active-inactive mode: The Drimmer is optionally automatically activated or inactivated. For example, the Drimmer may be inactivated when a patient's HR is above HRmax. HR is optionally calculated in real-time from the R-R interval, which is derived from a signal coming from lead E1 or E4. Alternatively, HR may be calculated from the mechanical signal coming from leads S1, S2, S3 or S4.

In some embodiments of the invention anti tachycardia features which are known in the art are optionally combined into the Drimmer.

The sensors S1-S4 acquire, optionally continuously, optionally in real-time, an A-D signal of the myocardium to which the sensors S1-S4 are connected. The sensors S1-S4 convey the signals of A-D to the IMD, where the signals are analyzed and processed.

From each A-D signal, from each site of a sensor, the time of rapid filling, Trf, is derived for a specific wall, at a specific heart cycle. Trf is optionally automatically determined from the A-D signal. A ratio between Trf and the time of diastole, Td, is defined, Ψ=Trf/Td, per cycle. Three values of Ψ may optionally be obtained: Ψcs, Ψs, Ψa. (for the CS, Septum (S), and apex respectively). In one form of operation, the greatest value of Ψcs, Ψs, Ψa, over a period to be defined is determined as Ψmx.

If Ψmx<Ψth (Ψth is a threshold), the IMD interprets the inequality as meaning that rapid LV filling ends early in diastole, and CO may be increased by pacing the RA.

Two more inputs are optionally used prior to pacing intervention. Pre-intervention mean HR, which must be <HRth (for example 100 bpm), and a level of patient's activity (asleep, exercising, etc).

The level of patient's activity is optionally determined from sensors of activity such as respiratory rate, or accelerometers indicating walking, jogging, etc. If pre-intervention HR<HRth, and patient is active, requires greater CO than baseline CO, then pacing procedure takes place. An example method for using sensory input and determining when to trim the diastole is provided below, with reference to the integration unit 430 of FIG. 4.

The IMD optionally calculates a rate most appropriate for a patient and will terminate the diastole with a pacing stimulus. The pacing stimulus is designed to activate the heart before the expected natural heart beat.

The pacing regimen attempts to be as physiological as possible. The pacing mechanism will increase HR by K % by pacing the RA. Pacing intervention will be controlled by Ψ obtained in real-time from the various walls and comparing it with Ψth.

In some embodiments of the invention, whenever the analysis shows that pacing is not needed it will be stopped.

In some embodiments of the invention, if pacing has increased CO, yet Ψ has remained <Ψth, then another increase of K % is optionally applied. In such a case, not only is CO increased, but the diastolic LV filling pattern is changed.

Other methods for controlling CO include having an upper limit of CO which is optionally allowed by the IMD, the value of which is optionally determined individually for each patient. In some embodiments of the invention, if the CO limit is reached, no further increase in HR is initiated by the IMD. The limitation is based on an understanding that increasing CO increases the consumption of myocardial oxygen.

In some embodiments of the invention, if HR increases as to exceed HRth, pacing intervention will stop.

In some embodiments of the invention, if sensors of activity detect a state of rest, pacing intervention will stop.

In some embodiments of the invention, the IMD analyzes the change of Ψ from the mean of Ψj, <Ψj>. The latter, ΔΨj, reflects a change in the mechanical property of ventricular wall j.

In another embodiment of the invention the mechanical properties of ventricular wall j is affected by an intervention.

In some embodiments of the invention, the sensor S6 measures CO in the RV, and provides input to the IMD with regard to the intervention of pacing. An optimal CO is determined from assessing activity level as described in the art.

In another embodiment of the invention the sensor S5 measures pressure in LA. If the pressure is greater than some threshold pressure, pacing is triggered. Additionally, values of concurrent diastolic parameters are examined by the IMD to evaluate whether increasing HR will increase CO. It is expected that increasing HR given a constant SV will increase CO and increase forward delivery of blood, thereby alleviating the backward pressure in the LA, thus preventing the clinical condition of pulmonary edema.

Chronic Atrial Fibrillation:

In DHF patients also having atrial fibrillation (AF), HR is irregular, that is, the R-R interval as optionally measured from lead E1 or E4 is irregular. Diastolic time varies with cycle length. The IMD optionally calculates Ψj for the various cycle lengths. It is expected that for the longer cycle lengths Ψj will be <Ψth. Therefore, the IMD will calculate real-time RR interval, and after waiting a predetermined time interval, determined from the measurement of RR intervals and Ψ, activates the lead E4 to pace the RV, thereby shortening cycle length and diastole respectively. This mode of action is expected to shorten futile prolonged diastole which does not provide better CO.

In some embodiments of the invention, CT scanning is synchronized with heart movement based on data from sensors used in the present invention. Presently, a CT machine scans the heart continuously. Off-line synchronization of images to the electric activity of the heart (specifically to the R wave peak) is performed. This creates a set of images all taken at a similar time instant within the cardiac cycle. However, there is limited success in synchronizing a desired mechanical cardiac event (diastasis) with an electric event (peak of the ECG R wave). Since some embodiments of the present invention can indicate specific time instants of diastole, for example the end of rapid filling and the beginning of the slow filling (diastasis), it is possible to synchronize CT imaging to this mechanical event (diastasis). This decreases the amount of radiation to which the patient is exposed, because CT images are taken only during diastasis and not during all cardiac cycle.

It is note that the synchronization may be performed by the implanted apparatus, such as described in FIGS. 1A, 1B, and 1C, and may be performed by a temporarily inserted device.

Additionally, some embodiments of the present invention can determine moments of least heart movement with the cardiac cycle, and send synchronizing signals to the CT system, to radiate the heart only during times of least movement, thereby saving the body from being exposed to unnecessary amounts of radiation. The embodiments of the present invention optionally provide the CT system an elapsed time within a heart cycle based, at least in part, on a value of one of the above-mentioned physiological parameters, and/or provide a synchronizing signal to the CTA system each instance of a specific time within the heart cycle, based, at least in part, on a value of one of the above-mentioned physiological parameters.

The physiologic understanding that cardiac output (CO) may be controlled by trimming the diastole, that is decreasing the volume of blood filling LV per cycle (SV per cycle) by artificial pacing (increasing HR), may be used in several other ways.

For example: it is possible to measure the velocity of a LV wall during diastole (e.g., by tissue Doppler imaging). Let VS represent the maximum velocity of the LV septum during diastole. It is possible to note whether VS=<VthS, where VthS is a predetermined velocity for which the rapid filling terminates early during diastole. A correlation exists between VS and the time, tS, it takes the septum to terminate its motion during diastole. In general, the smaller VS, the shorter is tS.

It is possible to calculate Ψs using tS and Td. Td depends linearly on HR (at least up to 100 cycles/min, where Td≅⅔ of cycle length). Parameter tS depends on VS, or on a filling pattern of LV, or on the grade of DD. Td is optionally calculated from knowing HR, and tS is estimated from knowing the grade of DD (from echocardiography).

Assuming that an average grade of DD is typically constant, for example grade II, estimate parameter tS. Td may be derived from knowing a patient's HR, which is optionally obtained from a standard pacemaker which keeps record of electric activity of the heart.

Under such circumstances, in some embodiments of the invention, a regular pacemaker is programmed as to pace the patient when tS/Td=Ψ, where Ψ<Ψth. In such embodiments, a “real-time” dialog between medical treatment and the pattern of LV filling exists with respect to the average mechanical behavior of the heart.

In some embodiments of the invention a pre-programming of a standard pacemaker yields good results when the grade of DD remains constant.

In some embodiments of the invention a table of values is constructed where for each VS and HR, a time delay from last electric activation is defined. Optionally, when the delay is completed, a next pacing stimulus is applied. Such a mode of action may allow standard pacemakers, lacking real-time measurements of LV filling characteristics, to improve CO “on the average” in patients with (1) an indication for a standard pacemaker due to some arrhythmia and (2) DHF.

In some embodiments of the invention a medical professional obtains a graph of LV volume over time (similar to FIGS. 3G and 3H) for a patient, and evaluates suitability of the patient for treatment with an embodiment of the invention based, at least in part, on the graph.

In some embodiments of the invention a medical professional obtains a graph of heart wall velocity over time (similar to FIG. 3A) and/or a graph of heart wall acceleration over time (similar to FIG. 3B) for a patient, for example by echocardiography, and evaluates suitability of the patient for treatment with an embodiment of the invention based, at least in part, on the graph.

In some embodiments of the invention a medical professional tries out trimming in a patient, optionally even a patient with a prior art pacemaker which is programmed to trim diastole. The medical professional causes the trimming to occur, and measures CO, for example by echocardiography.

Using such a method, diagnosis of diastolic heart failure is optionally made.

Using such a method, suitability of the patient to treatment by the Drimmer is optionally tried out by the medical professional.

Some more of the physiological basis underlying embodiments of the invention is now detailed.

An action of filling the heart with blood is a complex process. A simplified model of a mechanical spring is now used to explain current therapeutic concepts and some novel concepts concerned with embodiments of the present invention.

Consider the heart muscle as a spring. In DHF the spring (heart) may be stiffer than normal. Stretching the spring simulates the action of filling the heart with blood, whereas letting the spring recoil simulates heart contraction and the action of ejecting blood from the heart to circulation.

Current treatment of DHF is explained via two goals of treatment:

(1) Since the spring (heart) is stiffer than normal, more force is required to stretch it to a desired length. A greater filling pressure is required to stretch and distend the walls of the heart in order to fill the heart to a given volume.

Reference is now made to FIG. 3D, which is a simplified graph 250 illustrating effects of LV diastolic failure caused by decreased ventricular compliance on LV pressure-volume loop.

The graph 250 has an X-axis 251 showing LV volume, in units of milliliters, and a Y-axis 252 showing LV pressure, in units of mmHg.

A control loop 255 depicts an LV pressure-volume loop of a normal heart.

A second loop 256 depicts LV pressure-volume loop of a stiffer heart which has a decreased ventricular compliance.

A diastolic pressure-volume curve 258 for the normal heart is tangential to the LV pressure-volume loop of the normal heart.

A diastolic pressure-volume curve 259 for the stiffer heart is tangential to the LV pressure-volume loop of the stiffer heart.

The diastolic pressure-volume curve 259 for the stiffer heart shows that in order to fill the stiffer heart with a given volume of blood, say 100 ml, pressure in the ventricle must be greater than that in the normal heart, depicted by the diastolic pressure-volume curve 258 for the normal heart.

It is noted that the pressure in the left atrium (LA) must exceed that in the left ventricle (LV) to allow blood flow from the atrium to the ventricle.

Such a filling pressure may be clinically intolerable. In other words, if filling pressure exceeds a certain value, life threatening event may occur, such as pulmonary edema. Current treatment with diuretics is directed at reducing the volume of blood in the vessels and compartments of the heart, thereby lowering LV filling pressure.

The role of diuretic treatment is to move the pressure-volume curve, such as depicted in FIG. 3D to the left. In this case the LV is filled to a smaller end diastolic volume. Stroke volume (SV), which depends on end diastolic volume, is smaller as well. Pulmonary edema is prevented, at a price of a decrease in CO (which is the multiplication of SV and HR, which is not affected by diuretics).

(2) Since the spring (heart) is stiffer than normal, the time it takes to stretch it to the desired length (volume) is prolonged. If a cycle of stretching and releasing the spring is too short, there is not enough time for stretching to the desired length. In the heart, there is not enough time for filling the LV, to the desired volume. Prior art treatment with drugs such as beta-blockers slows down the cycle of stretching and releasing, thereby providing a longer time for LV filling.

Reference is now made to FIG. 3E, which depicts images of Doppler trans-mitral flow velocity in diastolic dysfunction grade I 261 and grade III 262.

In the image of the grade I diastolic dysfunction an E wave 264 representing the velocity of trans-mitral blood flow during early ventricular filling is decreased relative to an A wave 265 representing the velocity of trans-mitral blood flow during atrial contraction. Additionally, the shape of the E wave is triangular, where the duration of the increasing arm is short and the decreasing arm (arrow) is prolonged. The prolonged duration reflects a prolonged time required to stretch the stiffer spring, or to fill the stiffer LV with blood.

Comparing the heart muscle to a spring is simplistic. The stiffness of a simple spring is linear. The stiffness of the heart is nonlinear. The stiffness of the heart changes throughout diastole, as reported in Ohno et al. During approximately the first third, or first half, of diastole, stiffness is lowest, and then the stiffness increases. The difference in stiffness as a function of time of diastole is reflected by a rate of LV filling as a function of diastole (shown by Nakajima et al. and in FIG. 3F).

Reference is now made to FIG. 3F, which depicts graphs of percentage of LV volume as a function of time of diastole.

The left graph 271 of FIG. 3F depicts a percentage of LV volume as a function of time of diastole in patients with systemic sclerosis, and the right graph 272 of FIG. 3F depicts a percentage of LV volume as a function of time of diastole in a control group of patients.

The upper lines 274 275 in FIG. 3F depict the percentage of LV volume as a function of time of diastole.

The graphs are taken from Nakajima et al. Journal of Nuclear Medicine 2001; 42: pp. 183-188.

It is noted that LV filling patterns vary. In the left graph 271 most of the filling occurs in the first ⅓ of diastole, whereas in the right graph 272 most of the filling occurs toward the end of diastole. Systemic sclerosis is a rare example of DHF.

Reference is now made to FIG. 3G, which depicts graphs 281 282 comparing LV filling patterns in a normal heart and in DHF patients.

FIG. 3G depicts a difference in LV filling pattern in DHF patients versus the normal patients. A lower graph 282 depicts an LV filling pattern 284 of DHF patients superimposed on a normal LV filling pattern 285.

Arrows point out differences between the patterns. The rate of LV filling in DHF patients is increased early in diastole, and significantly decreases toward a latter part of the diastole. The arrows delineate transition points where the rate of LV filling changes. The change in rate of LV filling corresponds with the stiffness of LV. The increased rate of LV filling (the slope of LV volume versus time) early in diastole reflects the greater compliance of LV during this phase of filling. When LV is filled to a certain volume, stiffness of LV increases, and it is more difficult to fill the LV with blood, and therefore the rate of LV filling (slope after the arrows) decreases. It is evident that in DHF patients the rate of LV filling late in diastole is smaller than it is early in diastole.

Reference is now made to FIG. 3H, which includes a first graph 287 which depicts LV volume over time in a patient with DHF, and a second graph 288 which depicts LV volume over time in a patient with DHF to which pacing has been applied according to an example embodiment of the present invention.

FIG. 3H demonstrates that the pacing which has been applied according to an example embodiment of the present invention increase CO.

The first graph 287 includes an X-axis 289 of time, in units of mSec, and a Y-axis 290 of LV volume in units of percent of a maximum LV volume.

The second graph 288 includes an X-axis 291 of time, in units of mSec, and a Y-axis 292 of LV volume in units of percent of a maximum LV volume.

In the first graph 287 of FIG. 3H, three cycles of heart activity are given, specifically, LV volume 293 and an electrocardiogram (ECG) 294 are plotted versus time.

SV is estimated at 60 ml, and HR is 60 bpm, so the product of SV and HR results in a CO of 3600 ml/min.

The second graph 288 of FIG. 3H shows 4 cycles of heart activity. LV volume 295 and an ECG 296 are plotted versus time. Arrows 297 show timing of atrial stimuli which trim the diastole, activate the LV earlier, and increase HR. The earlier activation results in a smaller volume of blood filling the LV, because diastole is terminated earlier by pacing than it would have without the pacing. SV is reduced to 50 ml, compared with 60 ml prior to pacing. However, HR increases to 90 bpm instead of 60 bpm prior to pacing. The post-intervention CO (product of SV and HR) is 4500 ml/min compared with 3600 ml/min pre-intervention.

In some embodiments of the invention LV filling is evaluated in real time, to verify that CO post intervention is greater than CO pre-intervention.

Decreasing Diastolic LV Wall Stress

In some embodiments of the invention the Drimmer evaluates LV wall stress from LV motion data, and optionally decreases LV wall stress by decreasing LV end diastolic volume. The latter is optionally achieved by stopping before filling is complete, increasing HR, and in all pumping a greater volume per unit time from the heart to the circulation. By doing so, end diastolic volume (EDV) decreases, transferring the diastolic compliance curve (of FIG. 3D) to the left, to a lower pressure/lower volume area. See FIG. 3D compare the diastolic pressure-volume curve 258 for the normal heart to the diastolic pressure-volume curve 259 for the stiffer heart. Transferring the pressure-volume loop leftward and downward decreases diastolic LV wall stress, decreasing myocardial oxygen consumption.

In some embodiments of the invention, the Drimmer intervenes in the pacing, to decrease LV wall stress, optionally even if body needs for CO are not increased.

In some embodiments of the invention the Drimmer refrains from diastolic trimming for periods of rest, where the heart is allowed to fill at its own pace to maximum, so as not to train the heart to not fill completely

The following physiological observations are noted:

1. LV filling depends, in part, on the stiffness of LV.

2. Stiffness of the LV is a function of the stiffness of its components (LV walls).

3. Wall stiffness is non-linear throughout diastole, especially during the latter portion of diastole.

4. Stiffness may differ spatially, from wall to wall and within walls.

5. Wall stiffness may change due to extrinsic factors such as blood pressure (afterload), relation between right ventricle and LV, pericardial fluid, intrathoracic pressure, and so on.

6. Wall stiffness may change due to intrinsic factors such as elastic properties of the wall (including: amount of scar tissue after infarction, amount of collagen or its orientation in the myocardial tissue), and extent of blood perfusing the wall.

7. Modifiable intrinsic and/or extrinsic factors are identified for purpose of intervention. For example, insufficient blood perfusion causing ischemia (intrinsic factor) may be modified by revascularization.

8. Stretching LV to greater than normal volumes or increasing diastolic wall stress (as defined by Laplace equation) to a greater than normal values may elicit maladaptive processes leading to injury and death of myocardial tissue (see chapters 21, 22 in Braunwald's heart disease Edition 8, 2008). Therefore, it may be beneficial to prevent unnecessary LV stretch.

FIG. 3F above summarizes physiological events which the some embodiments of the invention aim to control and extract benefit for the DHF patient. FIG. 3F shows that the LV filling pattern of patients with systemic sclerosis (a form of DHF) differ from that of normal individuals. Some embodiments of the invention aim to detect the variability, optionally even in real-time, and to take advantage of the differences. Specifically, detecting that the LV filling pattern changes from the pattern in graph 272 of FIG. 3F to the pattern in graph 271 of FIG. 3F, based, or example, on the physiological inputs described for FIG. 1A. Such a change means that most of the LV filling occurs early in diastole. A portion of blood volume entering the LV during a later part of the diastole is relatively small. Waiting for the natural heart cycle to terminate, and LV filling to complete is less effective from the CO point of view, compared with earlier activation of the next cycle (increasing HR) on the expense of a smaller SV.

Real-time monitoring of diastolic LV wall characteristics optionally provides data for further intervention, optionally provided by embodiments of the invention, resulting in improved LV wall function; reduced wall stress; and optimizing CO. One such intervention is a “diastolic-property modifying technology”, which is part of some embodiments of the invention, and will be described below. The intervention aims to include myocardial stimulations which will modify the compliance of the myocardium.

Monitoring LV wall characteristics provides data for yet another intervention for improving LV wall function, and optionally optimizing CO. Such data correlated with metabolic and activity data may suggest, in some cases, that ischemia may be underlying LV wall dysfunction. The present invention, in some embodiments thereof, provides a way to assess real-time myocardial ischemia and a way to monitor whether coronary revascularization therapy was efficient.

For example, an LV filling pattern according to any of the sensed parameters may be recorded at a first point in time, such as an A-D pattern, a velocity pattern, a pressure pattern, a strain pattern, and so on. A second pattern of the same parameters may be recorded at a later time, and the two patterns compared. If the first pattern differs significantly from the first pattern, a development associated with myocardial ischemia may be suspected.

For example, the making of the first recording includes calculating a mean and a standard deviation of the physiological parameters during the first time; the making of the second recording includes calculating a mean and a standard deviation of the physiological parameters during the second time; and the determining includes determining whether differences between the first mean and the second mean are statistically significant.

For example, an LV filling pattern according to any of the sensed parameters may be recorded at a point in time, such as an A-D pattern, a velocity pattern, a pressure pattern, a strain pattern, and so on. A second pattern of the same parameters may be recorded at a later time, and the two patterns compared. If the first pattern is taken soon after coronary revascularization, and the second pattern taken some months later, differences between the first pattern and the second pattern indicate whether coronary revascularization therapy was successful over time.

It is noted that heart stiffness increases non-linearly with the increase in heart volume. Terminating diastole earlier by pacing results in a smaller end-diastolic volume and a smaller corresponding end-diastolic pressure. Thus, cardiac work which can be calculated from the pressure-volume loop is smaller in the range of smaller LV volumes. As a result LV wall stress (during diastole) is reduced and perfusion of transmural coronary arteries is improved. Thus, specific pacing intervention lowers the cardiac work per cycle, lowers the corresponding consumption of oxygen per cycle, and improves myocardial blood perfusion, while maintaining cardiac output.

In some embodiments of the invention the LV walls are treated differentially, according to data acquired from sensors measuring data related to the LV walls. The Drimmer, in some embodiments thereof, optionally intervenes with a specific LV wall which provides data requiring intervention.

There are two phases in early diastole which are energy-dependent and it is logical to influence them. One or both of the phases are influenced in some embodiments of the invention.

A first phase is isovolumic relaxation. For complete myocyte relaxation, cytosol must be largely cleared of calcium, so that calcium dissociates from troponin C, and tension-generating actin-myosin bonds must be lysed. Diastolic clearance of cytosolic calcium requires ATP to fuel the sarcoplasmic reticulum (SR) and sarcolemma (membrane of the myocyte) calcium-ATPases, which transport calcium into the SR or across the sarcolemma, respectively. Calcium removal also occurs via Na—Ca exchange, a process of secondary active transport driven by the low intracellular Na concentration.

In pathological conditions, for example when the myocardium is ischemic, or if digitalis preparation is used, intracellular calcium is increased and as a result the removal of calcium is incomplete. In some embodiments of the invention an electric stimulus, for example as described by Kieval in above-mentioned U.S. Pat. No. 5,800,464, to the tissue, myocytes, via an electrode is applied, rendering an outer surface of the membrane more negative than resting potential, at a precise timing relative to the isovolumic relaxation. This facilitates removal of calcium from the intracellular space by attracting the positive ions and facilitating their transport to the extracellular space.

In some embodiments of the invention, enhancing cardiac muscle relaxation is not limited to the specific description above. The enhancing is envisioned to operate with other forms of electric stimuli, via any form of electrode, applied to tissue for the purpose of enhancing cardiac muscle relaxation.

Some published material related to modification of cardiac contraction are published, many of them US patents to Ben Haim et al., listed in the “Field and Background of the Invention” section above. However the Ben Haim patents describe controlling mechanical systole, either enhancing or weakening the contraction by application of electric stimuli to the tissue. We emphasize that the stimulation provided according to this invention is timed after the T wave (of the ECG), and has no effect on the electric (systole) activation of the myocytes.

A second phase which is optionally modified is elastic recoil of the myocardium from its end systolic volume. Affecting the last part of the myocardial contraction results in a smaller end systolic volume, from which myocardium will recoil more forcefully, creating a stronger suction effect, thereby pumping more blood in the early part of diastole.

Information from the signals sensed by the sensors characterizes the stiffness of the LV walls. The stiffness of the LV walls is optionally correlated with activity parameters sensed by activity sensors. During physical exertion (walking, or heart rate >100 bpm) LV wall stiffness characteristics vary. Such variation may be related to insufficient blood supply to that part of the LV wall. The information, the relation between revascularization and LV mechanical characteristics may serve as an indication for future revascularization, and evaluation of successful revascularization.

In some embodiments of the invention wall stress is measured by one or more implantable biosensors attached to walls of the heart. In some embodiments of the invention wall pressure and/or strain and/or fluid flow are measured by one or more implantable biosensors attached to walls of the heart.

The information from the sensors is a different aspect, and may be more specific than stress testing or scintigraphy. An advantage of the sensor information stems from a direct “real-time” relation between LV wall mechanical function and the supplying coronary artery. This is in contrast to prior art stress testing, where ECG changes may reflect myocardial ischemia and other non-ischemic events (hypertension, LV wall stress).

A patient with DHF is prone to developing myocardial ischemia due to risk factors such as hypertension, diabetes mellitus, dyslipidemia, smoking cigarettes, and so on. Obtaining information regarding myocardial perfusion is important for such a patient. Such patients may have significant difficulties undergoing tests for demonstrating myocardial ischemia, for example stress testing, because of peripheral vascular disease, low general fitness, orthopedic or neurological problems. Scintigraphy is an option, however it is costly, and also here information is inferred from the perfusion scan. Coronary CTA is also an option relevant to patients with normal kidney function and who are not at risk for developing kidney failure.

In some embodiments of the invention, sensors provide data during the cardiac cycle. The data describes the cardiac cycle, and tracking the data over time can indicate changes in the heart. For example, A-D sensor data provides an LV filling pattern, such as depicted in FIGS. 3A and 3B. Tracking the filling pattern over time can show changes in heart function. For example, according to some embodiments of the invention, a patient who has had a stent implanted has filling patterns recorded soon after the implant, and again some time later (every few months). Changes in the filling pattern may indicate changes in heart function, without necessarily performing an angiogram, and optionally not even under a stress test. This is especially beneficial to patients which are not able to perform a standard stress test.

In some embodiments of the invention, a method is included for installing the sensors and the IMD. The method includes elements of echocardiography prior to installation, and measurements during installation. Optional echocardiography shows motion of the septum (S), coronary sinus (CS), apex (A), RV. Echocardiography assists in locating leads during a procedure of insertion of the apparatus (or during implantation).

Some Applications for Embodiments of the Present Invention

Some non-limiting examples of new applications for embodiments of the present invention include:

Treating patients with DHF of any cause (hypertension, LV hypertrophy (congenital or acquired), ischemic myocardium, LV outflow tract narrowing of any kind e.g. aortic stenosis, myocardial infiltrating diseases such as amyloidosis, systemic sclerosis).

Treating patients with atrial fibrillation (AF) also having DD. The patients may benefit from a specific pacing regimen which will improve LV filling and thereby increase CO. During AF some of the cardiac cycles may be prolonged. Applying an embodiment of the present invention optionally provides the patient with two outcomes: (a) solving the clinical manifestations of slow HR (dizziness, weakness, fainting, etc), and (b) optimizing CO. This is also a condition where diastolic LV wall stress is increased and may benefit from earlier pacing.

Treating patients with bradycardia of any cause also having DD may benefit from a pacing regimen that will improve LV filling and thereby increase CO. During bradycardia cardiac cycles may be prolonged. Applying some embodiments of this invention will provide the patient with two outcomes: (a) solving the clinical manifestations of slow HR (dizziness, weakness, fainting, etc), and (b) optimizing CO. This is in contrast to standard pacemakers which provide a solution only for the slow HR. This is also a condition where diastolic LV wall stress is increased and may benefit from earlier pacing.

In some embodiments of the invention, bradycardia is treated by measuring a time difference Tlast between two ventricular activations, measuring a time from a ventricular activations to an end of a rapid filling phase of LV, Trf, and if Trf/Tlast is smaller than threshold, producing a diastole ending signal.

In some embodiments of the invention, atrial fibrillation is treated by measuring a time difference Tlast between two ventricular activations, measuring a time from a ventricular activations to an end of a rapid filling phase of LV, Trf, and if Trf/Tlast is smaller than threshold, producing a diastole ending signal.

Providing a way to synchronize computed tomography angiography (CTA) scanning with the cycle of the heart. The problem: Cardiac CTA scans the heart, and in order to reconstruct the images of the heart, these images have to be scanned when the heart is at a same position and/or minimally moving. Today synchronization is limited by technology which is based on ECG “R” wave peak detection and then “selecting” the images of a predetermined time window relative to said “R” wave peak. This sometimes yields a good result, which is compromised when HR is irregular. Embodiments of the invention provide a way to determine the end of rapid filling phase, and even to provoke the end of rapid filling phase by suitable pacing, which is the preferred time for heart scanning using CTA. Embodiments of the invention offer a real-time dialog between a mechanical event in the heart and the CTA machine. A signal is transmitted from the communication module in the Drimmer (see FIG. 1) to the CT scanner, indicating a unique time for scanning the patient, optionally when the heart least moves, instead of a continuously, as performed today. Embodiments of the invention optionally provide for a prospective CT scanning of the human heart, using less X-ray radiation.

Some Indications for Use of Embodiments of the Invention:

A typical patient profile: An elderly patient, mean age of 74 years, with long standing hypertension, clinical symptoms of HF and recurrent hospitalizations for HF. Sinus rhythm on ECG. On echo: diastolic dysfunction, with good LV contraction (EF>50%), and a competent mitral valve. The typical patient, just described, is expected to display a relatively short rapid LV filling phase, due to a reduced compliance of the LV walls. Such a patient is typically treated with anti hypertensive drugs and HR slowing drugs, among which are beta blockers, and calcium channel blockers. It is therefore forecast that when such a patient exercises, he/she will find it difficult to increase HR, thereby increasing CO. The Drimmer can help such a patient by optional real-time analysis of the pattern of LV filling, verifying that the early portion of diastole ended “early”, and verifying that the latter portion of diastole contributes little to LV filling. If conditions are met, the Drimmer activates the patient's heart at a pace greater than the baseline pace.

Patients with other pathological conditions may benefit from embodiments of the invention in whole or in part. For example, a patient with an acquired atrioventricular (AV) block who require permanent pacing and also manifests symptoms of DHF may benefit from a standard pacemaker programmed as described above, resulting in two outcomes: (1) treatment of rate and conduction disturbances and (2) improving CO by pacing according to the description above in section: summary of the invention and as described for the typical patient. The patient with DHF who also suffers from the above conduction defect (AV block), typically depends on a pacemaker to provide HR. If a pacemaker's lower rate (the programmed baseline HR) is around 60 beats per minute, then this condition is quite similar to that of the typical patient described above. Similar because due to stiffness of LV walls, LV filling is limited, and due to pacemaker lower rate it is difficult to increase CO. The Drimmer optionally optimizes the timing of LV activation, optionally taking into consideration, not only time intervals between electric events, but optionally also mechanical events which may provide a way to increase CO.

The following examples show patients with other diseases which share common problems which the Drimmer can help solving. The patients suffer from DHF, that is stiffness of LV walls, and therefore from decreased stroke volume. The patients are dependent on external pacing. One specific solution is to pace these patients using standard pacemakers with an added programming based on an embodiment of the present invention, which solves the problem of HR. A different solution measures the pattern of LV filling, and under certain circumstances HR is increased to a greater rate in order to increase CO. Some example patients having these conditions are listed below, however beneficial use of embodiments of the invention is not limited to these conditions only: patients with an acquired third degree atriventricular block or advanced second degree associated with bradycardia with symptoms or periods of asystole of 3.0 seconds or any escape rate <40 beats/min without symptoms; patients who acquired third degree or advanced second degree AV block after cardiac surgery or patients with neuromuscular diseases with AV block; patients with medical conditions who require drugs that result in symptomatic bradycardia; patients after catheter ablation of the AV node.

Patients with chronic bifascicular and trifascicular block who require permanent pacing and also manifest symptoms of DHF may benefit from a standard pacemaker programmed as described above to result in two outcomes: (1) treatment of the rate and conduction disturbances and (2) improvement of CO by pacing according to the description above. For example: patients with intermittent third-degree AV block or type II second degree AV block, or alternating bundle branch block.

Patients who require permanent pacing after an acute phase of myocardial infarction due to conduction disturbances, and also manifest symptoms of DHF. For example: persistent second-degree AV block with bilateral bundle branch block.

Patients with sinus node dysfunction who require permanent pacing, and who also manifest symptoms of DHF.

Patients with various cardiomyopathies who require permanent pacing and also manifest symptoms of DHF. For example: a patient with hypertrophic cardiomyopathy with sinus node dysfunction or AV block, or patients who are medically refractory and symptomatic with significant resting or provoked LV outflow obstruction.

Patient with dilated cardiomyopathy with sinus node dysfunction or AV block, or patients who are treated with biventricular pacing who are also medically refractory and symptomatic and also manifest symptoms of DHF.

Patients who were resuscitated from ventricular fibrillation (VF) and require

Intra Cardiac Defibrillator (ICD) and also manifest symptoms of DHF.

It is expected that during the life of a patent maturing from this application many relevant types of heart pacers will be developed and the scope of the term heart pacer is intended to include all such new technologies a priori.

As used herein the term “approximately” refers to ±20%.

The terms “comprising”, “including”, “having” and their conjugates mean “including but not limited to”.

The term “consisting of” is intended to mean “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a unit” or “at least one unit” may include a plurality of units, including combinations thereof.

The words “example” and “exemplary” are used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. Apparatus for increasing cardiac output comprising:
a controller for producing a diastole ending signal; and
one or more leads connected to the controller, for carrying the signal to lead connections to a heart,
characterized by:
one or more physiological sensors connected to the controller and configured to provide input signals to the controller; and
the controller analyzing the input signals and detecting when a left ventricle (LV) of the heart is mostly full, and producing the diastole ending signal such that the diastole duration is shortened and cardiac output is increased.

2-7. (canceled)

8. Apparatus according to claim 1 in which the decision rules are based, at least in part, on a table which includes values of velocity of ventricular motion and heart rate, and a corresponding time delay from last electric activation to production of the diastole ending signal.

9. Apparatus according to claim 1 in which the controller is configured to refrain from producing the diastole ending signal, based, at least in part, on the input signals.

10-13. (canceled)

14. Apparatus according to claim 1 in which the input signals include a signal based, at least in part, whether atrial fibrillation is detected.

15. Apparatus according to claim 1 in which the input signals include a signal based, at least in part, on how much blood is in the left ventricle compared to a full left ventricle.

16. Apparatus according to claim 1 and further including one or more accelerometers, and in which the input signals include a signal based, at least in part, on acceleration measured at a wall of the heart.

17. Apparatus according to claim 1 in which the input signals include a signal based, at least in part, on wall tension measured at a wall of the heart.

18. Apparatus according to claim 1 in which the input signals include a signal based, at least in part, on oxygen saturation.

19. Apparatus according to claim 1 in which the input signals include a signal based, at least in part, on blood flow rate.

20-24. (canceled)

25. Apparatus according to claim 1 in which the input signals include a signal based, at least in part, on an input from a unit external to a patient's body.

26-27. (canceled)

28. Apparatus according to claim 1 and further comprising
a connection to a pacemaker,
characterized by the controller having decision rules for indicating to the pacemaker when to fire and end the diastole.

29. A method of programming a pacemaker to increase cardiac output characterized by trimming duration of diastole.

30. A method according to claim 29 and further comprising collecting data from one or more physiological sensors, and in which an amount by which the diastole duration is trimmed is based, at least in part, on analyzing the input signals and determining when a left ventricle of the heart is mostly full.

31. A method according to claim 29 and further comprising trimming the duration of the diastole based, at least in part, on a table which includes values of velocity of ventricular motion and heart rate, and a corresponding time delay from a last electric activation to production of a diastole ending signal.

32. A method for increasing cardiac output comprising:
producing a signal to trim diastole duration, thereby increasing heart rate (HR) and increasing a product of stroke volume (SV) times HR.

33. (canceled)

34. A method according to claim 32 and further comprising:
implanting a diastole trimming device in a patient's body; and
sensing one or more physiological parameters related to a body, and providing the one or more physiological parameters to a controller in the diastole trimming device.

35-37. (canceled)

38. A method according to claim 34 in which the controller is configured to refrain from trimming the diastole, based, at least in part, on the one or more physiological parameters.

39-42. (canceled)

43. A method according to claim 34 in which the one or more physiological parameters include a parameter based, at least in part, on how much blood is in the left ventricle compared to a full left ventricle.

44. A method according to claim 34 in which the one or more physiological parameters include a parameter based, at least in part, on acceleration measured at a wall of the heart.

45. (canceled)

46. A method according to claim 34 in which the one or more physiological parameters include a parameter based, at least in part, on oxygen saturation.

47. A method according to claim 34 in which the one or more physiological parameters include a parameter based, at least in part, on oxygen consumption.

48. A method according to claim 34 in which the one or more physiological parameters include a parameter based, at least in part, on blood flow rate.

49-53. (canceled)

54. A method according to claim 34 in which the one or more physiological parameters include a parameter based, at least in part, on an input from a unit external to a patient's body.

55. (canceled)

56. A method of treating diastolic heart failure comprising increasing cardiac output using the method of claim 32.

57-59. (canceled)

60. A method of treating atrial fibrillation comprising:
detecting ventricular activation;
measuring a time between last two ventricular activations, Tlast;
measuring a time to end of a rapid filling phase of LV, Trf;
if Trf/Tlast is smaller than threshold, producing a diastole ending signal.

61. A method of treating bradycardia comprising:
detecting ventricular activation;
measuring a time between last two ventricular activations, Tlast;
measuring a time to end of a rapid filling phase of LV, Trf;
if Trf/Tlast is smaller than threshold, producing a diastole ending signal.

62-63. (canceled)

64. A method for synchronizing computed tomography angiography (CTA) scanning with heart movement, comprising:
sensing a physiological parameter of the heart;
computing elapsed time within a heart cycle based, at least in part, on a value of the parameter; and
providing the elapsed time to a CTA system, in which the sensing, the computing, and the providing, are performed by an implanted apparatus.

65-67. (canceled)

68. A method for diagnosing a change in blood supply to a cardiac wall comprising:
making a first recording of one or more physiological parameters during LV filling cycle at a first time;
making a second recording of the same one or more physiological parameters during LV filling cycle at a second, later time;
comparing the first recording to the second recording; and
determining whether differences between the first recording and the second recording indicate a change in the blood supply to the cardiac wall.

69. (canceled)

70. A method for tracking cardiac revascularization comprising:
making a first recording of one or more physiological parameters during LV filling cycle soon after cardiac revascularization;
making a second recording of the same one or more physiological parameters during LV filling cycle later after cardiac revascularization;
comparing the first recording to the second recording; and
determining whether differences between the first recording and the second recording indicate a change in the efficacy of the cardiac revascularization.

71. Apparatus according to claim 1 in which the input signals include a signal based, at least in part, on one from a group comprising:
respiratory rate;
measured heart rate;
measured body motion;
a body's posture; and
a temperature.
